SEQUENCE LISTING (30 sequences)

SEQ ID NO: 1 - protein, 23 aa, Influenza virus
SLLTEVETPIRNEWGCRCNDSSD

SEQ ID NO: 2 - protein, 23 aa, Influenza virus
SLLTEVETPIRNEWGCKCNDSSD

SEQ ID NO: 3 - protein, 23 aa, Influenza virus
SLPTEVETPIRNEWGCRCNDSSD

SEQ ID NO: 4 - protein, 23 aa, Influenza virus
SLLTEVETPIRSEWGCRCNDSGD

SEQ ID NO: 5 - protein, 23 aa, Influenza virus
SFLTEVETPIRNEWGCRCNGSSD

SEQ ID NO: 6 - protein, 23 aa, Influenza virus
SLLTEVETPIRNEWECRCNGSSD

SEQ ID NO: 7 - protein, 23 aa, Influenza virus
SLLTEVETPTRNGWGCRCSDSSD

SEQ ID NO: 8 - protein, 23 aa, Influenza virus
SLLTEVETPIRNGWECRCNDSSD

SEQ ID NO: 9 - DNA, 432 bp, Homo sapiens
atgaagcacctgtggttcttcctcctgctggtggcggctcccagatgggtcctgtcccagctgcagctgcaggagtcgggcccaggactggtgaagccttcggagaccctgtccctcacctgcactgtctctggtggttccatcagcagtagtttttactactgtggctggatccgccagcccccagggaaggggctggagtggattgggagtatctattatcgtgggagcacctactacaacccgtccctcaagagtcgagtcaccatatccgtagacacgtccaagaaccagttctccctgaagctgagctctgtgaccgccgcagacacggctgtgtattactgtgcgagacgggttactatggttcggggagttaagggggactactttgactactggggccagggaaccctggtcaccgtctcctca

SEQ ID NO: 10 - DNA, 384 bp, Homo sapiens
atgagggtcctcgctcagctcctggggctcctgctgctctgtttcccaggtgccagatgtgacatccagatgacccagtctccatcctcactgtctgcatctgtaggagacagagtcaccatcacttgtcgggcgagtcagggtattagcagctggttagcctggtatcagcagaaaccagagaaagtccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataattattacccgctcactttcggcggagggaccaaggtggagatcaaacga

SEQ ID NO: 11 - protein, 144 aa, Homo sapiens
MKHLWFFLLLVAAPRWVLSQLQLQESGPGLVKPSETLSLTCTVSGGSISSSFYYCGWIRQPPGKGLEWIGSIYYRGSTYYNPSLKSRVTISVDTSKNQFSLKLSSVTAADTAVYYCARRVTMVRGVKGDYFDYWGQGTLVTVSS

SEQ ID NO: 12 - protein, 128 aa, Homo sapiens
MRVLAQLLGLLLLCFPGARCDIQMTQSPSSLSASVGDRVTITCRASQGISSWLAWYQQKPEKVPKSLIYAASSLQSGVPSRFSGSGSGTDFTLTISSLQPEDFATYYCQQYNYYPLTFGGGTKVEIKR

SEQ ID NO: 13 - DNA, 31 bp, Artificial Sequence
  Description of Artificial Sequence Primer
tcttgtccaccttggtgttgctgggcttgtg

SEQ ID NO: 14 - DNA, 26 bp, Artificial Sequence
  Description of Artificial Sequence Primer
gttgaagctctttgtgacgggcgagc

SEQ ID NO: 15 - DNA, 23 bp, Artificial Sequence
  Description of Artificial Sequence Primer
ggtgccagggggaagaccgatgg

SEQ ID NO: 16 - DNA, 30 bp, Artificial Sequence
  Description of Artificial Sequence Primer
aggcacacaacagaggcagttccagatttc

SEQ ID NO: 17 - DNA, 27 bp, Artificial Sequence
  Description of Artificial Sequence Primer
ggtccgggagatcatgagggtgtcctt

SEQ ID NO: 18 - DNA, 19 bp, Artificial Sequence
  Description of Artificial Sequence Primer
gatttaggtgacactatag

SEQ ID NO: 19 - DNA, 20 bp, Artificial Sequence
  Description of Artificial Sequence Primer
taatacgactcactataggg

SEQ ID NO: 20 - DNA, 44 bp, Artificial Sequence
  Description of Artificial Sequence Primer
agagagagagatctctcaccatgagggtcctcgctcagctcctg

SEQ ID NO: 21 - DNA, 34 bp, Artificial Sequence
  Description of Artificial Sequence Primer
ctctctctcgtacgtttgatctccaccttggtcc

SEQ ID NO: 22 - DNA, 40 bp, Artificial Sequence
  Description of Artificial Sequence Primer
agagagaggtcgacaccatgaagcacctgtggttcttcct

SEQ ID NO: 23 - DNA, 33 bp, Artificial Sequence
  Description of Artificial Sequence Primer
ctctctctgctagctgaggagacggtgaccagg

SEQ ID NO: 24 - DNA, 27 bp, Artificial Sequence
  Description of Artificial Sequence Primer
ggtacgtgaaccgtcagatcgcctgga

SEQ ID NO: 25 - DNA, 27 bp, Artificial Sequence
  Description of Artificial Sequence Primer
tctatataagcagagctgggtacgtcc

SEQ ID NO: 26 - DNA, 30 bp, Artificial Sequence
  Description of Artificial Sequence Primer
ccaagggcccatcggtcttccccctggcac

SEQ ID NO: 27 - DNA, 24 bp, Artificial Sequence
  Description of Artificial Sequence Primer
gacaccctcatgatctcccggacc

SEQ ID NO: 28 - DNA, 24 bp, Artificial Sequence
  Description of Artificial Sequence Primer
cgacatcgccgtggagtgggagag

SEQ ID NO: 29 - DNA, 24 bp, Artificial Sequence
  Description of Artificial Sequence Primer
tgttctccggctgcccattgctct

SEQ ID NO: 30 - DNA, 26 bp, Artificial Sequence
  Description of Artificial Sequence Primer
tggctgcaccatctgtcttcatcttc

(19) United States
(12) Patent Application Publication
(10) Pub. No.: US 2003/0219442 A1
(43) Pub. Date: Nov. 27, 2003

(54) Human monoclonal antibodies to influenza M2 protein and methods of making and using same

(76) Inventors: Toshifumi Mikayama, Takasaki (JP); Rongfang Wang, San Diego, CA (US); Shinichiro Kato, San Diego, CA (US); Hilde Cheroutre, Del Mar, CA (US)

Correspondence Address: Pillsbury Winthrop LLP, Intellectual Property Group, P.O. Box 10500, McLean, VA, 22102, US

(21) Appl. No.: 10/389,221

(22) Filed: Mar. 13, 2003

Related U.S. Application Data
(60) Provisional application No. 60/364,997, filed on Mar. 13, 2002.

(51) Int. Cl. C12Q 1/70; C07H 21/04; A61K 39/42; C12P 21/02; C12N 5/06; C07K 16/08

(52) U.S. Cl. 424/159.1; 435/5; 435/69.1; 435/320.1; 530/388.3; 536/23.53; 435/339

(57) ABSTRACT

Human, humanized and chimeric monoclonal antibodies that bind to influenza M2 protein. The antibodies are useful for, among other things, treatment, diagnostics, purifying and isolating M2 or influenza virus, and identifying the presence of M2 or influenza virus in a sample or a subject.

### PRIORITY APPLICATION INFORMATION

This application claims priority to U.S. Provisional Application Serial No. 60/364,997, filed Mar. 13, 2002.

### TECHNICAL FIELD

The invention relates to antibodies, more particularly to human, humanized and chimeric antibodies that specifically bind to influenza virus M2 protein.

### BACKGROUND

Influenza types A or B viruses cause epidemics of disease almost every winter in all countries and are a leading cause of death in the developed world. In the United States, these winter influenza epidemics can cause illness in 10% to 20% of people and are associated with an average of 20,000 deaths and 114,000 hospitalizations per year. The present strategy for control of influenza is yearly vaccination with inactivated whole-virus or sub-unit vaccines. The major neutralizing antigen of the influenza virus is hemagglutinin (HA) (Frace et al., Vaccine 17:2237 (1999)). However, due to frequent and unpredictable antigenic variation of HA, the vaccine frequently fails to provide optimal protective immunity against divergent viral strains. Moreover, for immuno-compromised individuals such as elderly patients, cancer patients and other patients who are immuno-incompetent due to ongoing treatment and/or disease, vaccination may not provide effective protection.

Hemagglutinin (HA) and neuraminidase (NA) are the two major antigens for the stimulation of antibody production. Due to frequent antigenic variation of these two proteins, they do not represent optimal targets for development of therapeutic drugs. A third transmembrane protein of type A influenza viruses, matrix protein 2 (M2), is abundantly expressed by virus-infected cells, where it is postulated to provide an obligatory transmembrane proton flux for viral replication (Ciampor et al., Virus Research 22:247 (1992); Grambas and Hay, Virology 190:11 (1992); Sugrue et al., EMBO Journal 9:3469 (1990)). Unlike HA and NA, M2 is conserved and may represent a target for the development of antibody-based passive immunotherapies for influenza patients (Ito et al., J. Virology 65:5491 (1991); Slepushkin et al., Vaccine 13:1399 (1995); Neirynck et al., Nature Med. 5:1157 (1999)).

Vaccination of mice with baculovirus-expressed M2 protein has been reported to enhance clearance of virus from mouse lungs and protect mice from a lethal challenge with both homologous and heterologous influenza A viruses (Slepushkin et al., Vaccine 13:1399 (1995)). A more recent report has shown that the fusion of the extracellular domain of M2 to the hepatitis B virus core (HBc) protein to create a fusion gene coding for M2HBc, when used as a vaccine could provide 90-100% protection against a lethal virus challenge in mice (Neirynck et al., Nature Med. 5:1157 (1999)). This protection could be passively transferred to unvaccinated mice using serum from M2HBc vaccinated mice. Zebedee et. al. demonstrated that an anti-M2 mouse monoclonal antibody had a moderate effect on the growth of influenza virus in a plaque assay. The size of the plaques, but not the number of plaques, for the A/Udorn/72 virus was smaller when the antibody was present during incubation. No effect was observed on the size or number of plaques for the A/WSN/33 strain indicating that this particular monoclonal antibody is not broadly effective against different influenza strains (Zebedee and Lamb, J. Virol 62:2762 (1988)). When this antibody was passively transferred to mice one day before viral challenge, the level of virus replication in the lungs 3 to 4 days after infection was approximately 100-fold less than that in animals receiving an irrelevant antibody (Treanor et al., J. Virol 64:1375). However, when this antibody was administered to SCID mice one day before virus infection, lung virus titers were no different from control mice (Palladino et al., J. Virol. 69:2075 (1995)). Mozdzanowska et. al. (Virology 254:138 (1999) using the same murine anti-M2 monoclonal antibody, 14C2, was able to demonstrate, in agreement with Zebeedee et. al, that an anti-M2 monoclonal antibody can reduce virus titers in a viral plaque assay but was unable to reduce viral titer of influenza strain A/PR/8/34 indicating that 14C2 does not broadly protect against influenza.

### SUMMARY

Fully human, humanized and chimeric (e.g., human/mouse chimera) anti-M2 monoclonal antibodies disclosed herein can recognize the A/PR/8134 and A/HK/8/68 strains indicating broad reactivity against influenza A. Furthermore, human, humanized and chimeric anti-M2 monoclonal antibody disclosed herein can protect mice from a lethal challenge of the A/PR/8/34 influenza A strain when the antibody is administered after the animals have been infected with influenza A.

The invention therefore provides compositions including human, humanized and chimeric antibodies that bind to influenza virus protein M2, pharmaceutical compositions containing human, humanized and chimeric antibody and kits containing the antibody. The human, humanized and chimeric antibodies of the invention are useful for treating influenza in a subject having or at risk of having influenza, including before infection (prophylaxis) or following infection (therapeutic); influenza diagnostics, including measuring virus titre; purification/isolation including purifying or isolating whole virus or M2 protein; and other assay systems. The invention therefore also provides methods of using the antibodies in therapy (e.g., treatment of influenza infection), diagnostics (detecting amounts of influenza or M2 protein in a sample) and purification (purifying or isolating influenza virus or M2 protein).

In one embodiment, a human antibody that specifically binds to at least a part of the M2 extracellular domain is provided. In a particular aspect, the extracellular domain comprises the amino acid sequence SLLTEVETPIRNEWGCRCNDSSD (SEQ ID NO: 1), a subsequence thereof or an amino acid variant thereof (e.g., an amino acid substitution, insertion, deletion or addition). In another aspect, the amino acid substitution is selected from:

| | |
|---|---|
| SLLTEVETPIRNEWGCKCNDSSD, SLPTEVETPIRNEWGCRCNDSSD, | (SEQ ID NOS:2-8, respectively) |
| SLLTEVETPIRSEWGCRCNDSGD, SFLTEVETPIRNEWGCRCNGSSD, | |
| SLLTEVETPIRNEWECRCNGSSD, SLLTEVETPTRNGWGCRCSDSSD, or | |
| SLLTEVETPIRNGWECRCNDSSD | |

Antibodies of the invention include polyclonal and monoclonal antibodies and mixtures thereof, which can be any of IgG, IgA, IgM, IgE, IgD, and any isotype thereof, for example, IgG₁, IgG₂, IgG₃ or IgG₄. Antibodies include intact human, humanized and chimeric immunoglobulin molecules with two full-length heavy chains and two full-length light chains (e.g., heavy and light chain variable regions) as well as subsequences of heavy or light chain which retain at least a part of a function (M2 binding specificity, M2 binding affinity or anti-influenza virus activity) of parental intact human, humanized and chimeric antibody that specifically binds M2. Exemplary subsequences include Fab, Fab′, (Fab′)₂, Fv, Fd, single-chain Fvs (scFv), disulfide-linked Fvs (sdFv) and V_{L} or V_{H}, or other M2 protein binding fragment of an intact human or humanized immunoglobulin. Antibodies of the invention therefore include heavy-chain variable sequence and light-chain variable sequence of the antibody produced by the hybridoma or a CHO cell line denoted as no. 2074 (ATCC PTA-4025), 161 (ATCC PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003) and L17 (ATCC Deposit No.; American Type Culture Collection, Manassas, Va., USA).

In various aspects, the antibody is produced by a cell line (e.g., a hybridoma or a CHO cell line) denoted as no. 2074 (ATCC Deposit No. PTA-4025; American Type Culture Collection, Manassas, Va., USA), 161(ATCC Deposit No. PTA-4026; American Type Culture Collection, Manassas, Va., USA), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003) and L17 (ATCC Deposit No.; American Type Culture Collection, Manassas, Va., USA).

Antibodies further include human, humanized and chimeric antibodies having the binding specificity and binding affinity of the human, humanized and chimeric antibodies of the invention. In one embodiment, an antibody has the binding specificity of an antibody produced by a cell line (e.g., a hybridoma or a CHO cell line) denoted as no. 2074 (ATCC Deposit No. PTA-4025; American Type Culture Collection, Manassas, Va., USA), 161(ATCC Deposit No. PTA-4026; American Type Culture Collection, Manassas, Va., USA), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003) and L17 (ATCC Deposit No.; American Type Culture Collection, Manassas, Va., USA). In another embodiment, an antibody has the binding affinity of an antibody produced by a cell line (e.g., a hybridoma or a CHO cell line) denoted as no. 2074 (ATCC Deposit No. PTA-4025; American Type Culture Collection, Manassas, Va., USA), 161(ATCC Deposit No. PTA-4026; American Type Culture Collection, Manassas, Va., USA), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003) and L17 (ATCC Deposit No.; American Type Culture Collection, Manassas, Va., USA).

Antibodies of the invention additionally include human, humanized and chimeric antibodies having the ability to inhibit virus infection in vitro or in vivo or that inhibit M2 binding of a cell, as the exemplified antibodies produced by a cell line (e.g., a hybridoma or a CHO cell line) denoted as no. 2074 (ATCC Deposit No. PTA-4025; American Type Culture Collection, Manassas, Va., USA), 161 (ATCC Deposit No. PTA-4026; American Type Culture Collection, Manassas, Va., USA), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003) and L17 (ATCC Deposit No.; American Type Culture Collection, Manassas, Va., USA). In one embodiment, an antibody has an EC₅₀ less than 3.0 μg/ml for inhibiting influenza virus infection of MDCK cells, as determined by a cell based-ELISA assay. In various aspects, the influenza virus is influenza A virus, such as A/PR/8/34 or A/HK8/68.

Antibodies of the invention further include human, humanized and chimeric antibodies that bind to two or more M2 proteins having different amino acid sequences, which may optionally be present on different influenza viruses (e.g., strains or isolates). In one embodiment, the antibody binds to at least a part of an M2 extracellular domain sequence. In a particular aspect, an M2 extracellular domain sequence includes the amino acid sequence SLLTEVETPIRNEWGCRCNDSSD (SEQ ID NO: 1), a subsequence thereof or an amino acid variant thereof (e.g., an amino acid substitution, insertion, deletion or addition), such as SLLTEVETPIRNEWGCKCNDSSD (SEQ ID NO: 2). In another particular aspect, an M2 extracellular domain sequence is selected from: SLLTEVETPIRNEWGCKCNDSSD, SLPTEVETPIRNEWGCRCNDSSD, SLLTEVETPIRSEWGCRCNDSGD, SFLTEVETPIRNEWGCRCNGSSD, SLLTEVETPIRNEWECRCNGSSD, SLLTEVETPTRNGWGCRCSDSSD, and SLLTEVETPIRNGWECRCNDSSD (SEQ ID NOS: 2-8, respectively).

Antibodies of the invention include those that have been modified to form oligomers, e.g., through the attachment of as oligomerization domain (e.g., leucine zipper motif) or via a cross-linking agent (e.g., chemical cross linker). Thus, antibodies of the invention include multimeric forms, for example, dimers, trimers, tetramers or higher order human, humanized and chimeric antibody oligomers. Such antibody multimers typically exhibit increased avidity for M2 in comparison to monomeric antibody.

Antibodies of the invention further include one or more heterologous domains that impart a distinct function or activity on a human or humanized antibody that binds M2. Antibodies that include an amino acid heterologous domain when one or more amino acids are distinct from the antibody (i.e., they are not a part of the native antibody). In one embodiment, a heterologous domain comprises a binding protein (e.g., receptor or ligand binding), an enzyme activity, a drug, an antiviral, a toxin, an immune-modulator, a detectable moiety or a tag. In one aspect, the binding protein comprises an antibody having a different binding specificity or affinity than human, humanized or chimeric antibody that specifically binds to influenza protein M2. Thus, the invention further provides multi-specific and multi-functional antibodies (e.g., bispecific and bifunctional antibodies, such as antibodies that bind to two or more antigens or that have two or more functions or activities, respectively).

Antibodies of the invention can bind to influenza protein M2, optionally present on one or more influenza strains or isolates. Thus, the antibodies have one or more effects on M2 or influenza virus infectivity, replication, proliferation, titre, severity or duration of one or more symptoms or complications associated with influenza, or susceptibility of influenza virus infection, i.e., anti-influenza virus activity. In one embodiment, a human, humanized or chimeric antibody inhibits infection of a cell in vitro or in vivo by one or more influenza strains or isolates. In another embodiment, a human, humanized or chimeric antibody reduces influenza virus titer or an amount of an influenza viral protein of one or more influenza strains or isolates. In yet another embodiment, a human, humanized or chimeric antibody inhibits or prevents increases in influenza virus titer or an amount of an influenza viral protein of one or more influenza strains or isolates. In still another embodiment, a human, humanized or chimeric antibody protects a subject from infection or decreases susceptibility of the subject to infection by one or more influenza strains or isolates. In a further embodiment, a human, humanized or chimeric antibody decreases one or more symptoms or complications associated with infection by one or more influenza strains or isolates (e.g., chills, fever, cough, sore throat, nasal congestion, sinus congestion, nasal infection, sinus infections body ache, head ache, fatigue, pneumonia, bronchitis, ear infection or ear ache). In various aspects, human, humanized or chimeric antibody is administered systemically (e.g., intravenous injection, subcutaneous injection, intravenous infusion, intramuscular injection), or locally to mucosal tissue (e.g., nasal passages, sinuses, throat, larynx, esophagus, ear or ear canal) or lung of a subject. In various aspects, the influenza strain is selected from A/PR/8/34 or A/HK/8/68, or other strains selected from H1N1, H2N2, H3N2, H5N1, H9N2, H2N1, H4N6, H6N2, H7N2, H7N3, H4N8, H5N2, H2N3, H11N9, H3N8, H1N2, H11N2, H11N9, H7N7, H2N3, H6N1, H13N6, H7N1, H11N1, H7N2 and H5N3.

Host cells that express invention human, humanized and chimeric antibodies are also provided. Cells include but are not limited to bacteria, yeast, plant, animal (e.g., mammalian cells such as hybridoma cell lines and CHO cell lines) as well as whole organisms such as non-human animals and plants that express an invention human, humanized or chimeric antibody.

Nucleic acids encoding the invention antibodies, including subsequences and variants thereof, are further provided. Nucleic acids include vectors for cloning or other genetic manipulation of the nucleic acid or for expression in solution, in a cell, or in any organism.

Combination compositions including antibodies of the invention are also provided. In one embodiment, a composition includes human, humanized or chimeric antibody that binds influenza M2 protein and an antiviral agent. In another embodiment, a composition includes a human, humanized or chimeric antibody that binds influenza M2 protein and an agent that inhibits one or more symptoms or complications associated with influenza infection (e.g., chills, fever, cough, sore throat, nasal congestion, body ache, head ache, fatigue, pneumonia, bronchitis, sinus infection or ear infection).

Pharmaceutical compositions including invention antibodies and a pharmaceutically acceptable carrier or excipient are provided. In one embodiment, a carrier is suitable for administration to mucosal tissue (e.g., nasal passages, sinuses, throat, larynx, esophagus) or lung of a subject.

Kits that include one or more invention antibodies in a container are also provided. In one embodiment, a kit includes instructions for treating (prophylaxis or therapeutic), inhibiting, preventing, decreasing susceptibility to, or reducing one or more symptoms or complications associated with influenza virus infection of a subject by one or more influenza strains or isolates. In another embodiment, the container comprises an aerosol, spray or squeeze bottle suitable for inhalation or nasal administration to a subject. In yet another embodiment, the kit or container includes an antiviral agent (e.g., an antibody or a drug) or an agent that inhibits one or more symptoms or complications associated with influenza infection.

Methods for treating influenza infection of a subject are provided. In one embodiment, a method includes administering to the subject an amount of a human, humanized or chimeric antibody that specifically binds influenza M2 effective to treat influenza infection of the subject. In one aspect, the antibody is administered substantially contemporaneously with or following infection of the subject, i.e., therapeutic treatment. In another aspect, the antibody provides a therapeutic benefit. In various aspects, a therapeutic benefit includes reducing or decreasing one or more symptoms or complications of influenza infection, virus titer, virus replication or an amount of a viral protein of one or more influenza strains. Symptoms or complications of influenza infection that can be reduced or decreased include, for example, chills, fever, cough, sore throat, nasal congestion, sinus congestion, nasal infection, sinus infection, body ache, head ache, fatigue, pneumonia, bronchitis, ear infection or ear ache. In still another aspect, a therapeutic benefit includes hastening a subject's recovery from influenza infection.

Methods for inhibiting infection of a subject by one or more influenza strains or isolates are also provided. In one embodiment, a method includes administering to the subject an amount of a human, humanized or chimeric antibody that specifically binds influenza M2 effective to inhibit infection of the subject or reduce susceptibility of the subject to influenza infection by one or more influenza strains or isolates. In various aspects, the antibody is administered prior to (prophylaxis), substantially contemporaneously with or following infection of the subject. In another aspect, the antibody provides a therapeutic benefit. In various aspects, a therapeutic benefit includes reducing or decreasing one or more symptoms or complications of influenza infection (e.g., chills, fever, cough, sore throat, nasal congestion, sinus congestion, nasal infection, sinus infection, body ache, head ache, fatigue, pneumonia, bronchitis, ear infection or ear ache), virus titer or an amount of a viral protein of one or more influenza strains or isolates, or susceptibility of a subject to infection by one or more influenza strains or isolates.

Methods for preventing an increase in influenza virus titer, virus replication, virus proliferation or an amount of an influenza viral protein in a subject are further provided. In one embodiment, a method includes administering to the subject an amount of a human, humanized or chimeric antibody that specifically binds influenza M2 effective to prevent an increase in influenza virus titer, virus replication or an amount of an influenza viral protein of one or more influenza strains or isolates in the subject.

Methods for protecting a subject from infection or decreasing susceptibility of a subject to infection by one or more influenza strains or isolates are additionally provided. In one embodiment, a method includes administering to the subject an amount of a human, humanized or chimeric antibody that specifically binds influenza M2 effective to protect the subject from infection, or effective to decrease susceptibility of the subject to infection, by one or more influenza strains or isolates. In one aspect, the protection includes reducing or decreasing one or more symptoms or complications associated with influenza infection (e.g., chills, fever, cough, sore throat, nasal congestion, sinus congestion, nasal infection, sinus infection, body ache, head ache, fatigue, pneumonia, bronchitis, ear infection or ear ache).

Methods of the invention can be practiced with antibody having the binding specificity or binding affinity of an antibody produced by a cell line (e.g., a hybridoma or a CHO cell line) denoted as no. 2074 (ATCC Deposit No. PTA-4025; American Type Culture Collection, Manassas, Va., USA), 161 (ATCC Deposit No. PTA-4026; American Type Culture Collection, Manassas, Va., USA), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003) and L17 (ATCC Deposit No.; American Type Culture Collection, Manassas, Va., USA). Antibodies can be included in a pharmaceutically acceptable carrier or excipient prior to administration to a subject.

Methods of the invention, including therapeutic, diagnostic and purification/isolation are applicable to any influenza strain/isolate or combination of strains/isolates. In various embodiments, the influenza strain is selected from A/PR/8/34 or A/HK/8/68, or other strains selected from H1N1, H2N2, H3N2, H5N1, H9N2, H2N1, H4N6, H6N2, H7N2, H7N3, H4N8, H5N2, H2N3, H11N9, H3N8, H1N2, H11N2, H11N9, H7N7, H2N3, H6N1, H13N6, H7N1, H11N1, H7N2 and H5N3.

### DESCRIPTION OF DRAWINGS

FIG. 1 illustrates nucleotide and amino acid sequences of variable region of immunoglobulin light chain of C40 antibody (C40Lv (SEQ ID NO: 10)) and of heavy chain (C40Hv (SEQ ID NO: 9)).

FIG. 2 shows that antibody nos. 2074, N547, L66 and C40G1 bind to M2 on A) A/PR/8/34 and B) A/HK/8/68 virus infected MDCK cells.

FIG. 3 shows a comparison of protective efficacy of A) C40G1, C40G4 and L30; and B) no. 2074, F1 and F2 antibodies, and that IgG1 isotype M2 antibodies provide greater protection of animals from a lethal virus challenge than antibodies with weak binding affinity to M2 on viral infected MDCK cells (i.e. F1 and F2).

FIG. 4 illustrates a comparison of M2 antibody binding to A) M2 peptide/BSA and B) M2 expressed on influenza virus infected cells.

FIG. 5 shows prophylactic protection of animals administered M2 antibody no. 2074.

FIG. 6 shows therapeutic protection of animals administered M2 antibody no. 2074.

### DETAILED DESCRIPTION

The invention is based at least in part on human, humanized and chimeric anti-M2 monoclonal antibodies. Several of the invention antibodies have broad reactivity against various M2 extracellular domain sequences based upon divergent influenza A virus strains. Passive transfer of an invention human anti-M2 monoclonal antibody protected animals from a lethal dose challenge of influenza A/PR/8/34, in both prophylactic (prior to virus infection) and therapeutic (following virus infection) mouse influenza models. Antibodies of the invention are therefore useful for treating a broad array of influenza strains or isolates. In addition, since invention antibodies are human they are less likely to induce hypersensitivity from repeated administration and are more likely to remain in a subjects' (e.g., a human) body for a longer period of time.

Thus, in accordance with the invention, there are provided human, humanized and chimeric antibodies that specifically bind to influenza M2 protein. In one embodiment, a human, humanized or chimeric antibody that specifically binds to influenza protein M2 extracellular domain is provided. In a particular aspect, an extracellular domain comprises the amino acid sequence SLLTEVETPIRNEWGCRCNDSSD (SEQ ID NO: 1), a portion thereof or an amino acid variant thereof (e.g., an amino acid substitution, insertion, deletion or addition), such as SLLTEVETPIRNEWGCKCNDSSD (SEQ ID NO: 2). In particular aspects, an extracellular domain having an amino acid substitution is selected from: SLLTEVETPIRNEWGCKCNDSSD, SLPTEVETPIRNEWGCRCNDSSD, SLLTEVETPIRSEWGCRCNDSGD, SFLTEVETPIRNEWGCRCNGSSD, SLLTEVETPIRNEWECRCNGSSD, SLLTEVETPTRNGWGCRCSDSSD, and SLLTEVETPIRNGWECRCNDSSD (SEQ ID NOS: 2-8, respectively).

The term “antibody” refers to a protein that binds to other molecules (antigens) via heavy and light chain variable domains, V_{H} and V_{L}, respectively. “Antibody” refers to any immunoglobulin molecule, such as IgM, IgG, IgA, IgE, IgD, and any subclass thereof. The term “antibody” also means a functional fragment of immunoglobulin molecules, such as Fab, Fab′, (Fab′)₂, Fv, Fd, scFv and sdFv, unless otherwise expressly stated.

The terms “M2 antibody” or “anti-M2 antibody” means an antibody that specifically binds to influenza M2 protein. Specific binding is that which is selective for an epitope present in M2 protein. That is, binding to proteins other than M2 is such that the binding does not significantly interfere with detection of M2. Selective binding can be distinguished from non-selective binding using assays known in the art.

Exemplary antibodies of the invention are denoted as no. 2074 (ATCC Deposit No. PTA-4025), 161(ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos., respectively; American Type Culture Collection, Manassas, Va., USA). Exemplary heavy-chain variable sequence and light-chain variable sequence is an amino acid sequence set forth in SEQ ID NO: 11 and SEQ ID NO: 12, respectively.

As used herein, the term “monoclonal,” when used in reference to an antibody, refers to an antibody that is based upon, obtained from or derived from a single clone, including any eukaryotic, prokaryotic, or phage clone. A “monoclonal” antibody is therefore defined herein structurally, and not the method by which it is produced. As used herein, a specific name, numeral or other designation given to a hybridoma or other cell line, such as no. 2074, 161, N547, L66 and C40G1, also is used to refer to the name of antibody.

The term “human” when used in reference to an antibody, means that the amino acid sequence of the antibody is fully human. A “human M2 antibody” or “human anti-M2 antibody” therefore refers to an antibody having human immunoglobulin amino acid sequences, i.e., human heavy and light chain variable and constant regions that specifically bind to M2. That is, all of the antibody amino acids are human or exist in a human antibody. Thus, for example, an antibody that is non-human may be made fully human by substituting the non-human amino acid residues with amino acid residues that exist in a human antibody. Amino acid residues present in human antibodies, CDR region maps and human antibody consensus residues are known in the art (see, e.g., Kabat, Sequences of Proteins of Immunological Interest, 4^{th} Ed.US Department of Health and Human Services. Public Health Service (1987); and Chothia and Lesk J. Mol. Biol. 186:651 (1987)). A consensus sequence of human V_{H} subgroup III, based on a survey of 22 known human V_{H} III sequences, and a consensus sequence of human V_{L} kappa-chain subgroup I, based on a survey of 30 known human kappa I sequences is described in Padlan Mol. Immunol. 31:169 (1994); and Padlan Mol. Immunol. 28:489 (1991)).

The term “humanized” when used in reference to an antibody, means that the amino acid sequence of the antibody has non-human amino acid residues (e.g., mouse, rat, goat, rabbit, etc.) of one or more determining regions (CDRs) that specifically bind to the desired antigen (e.g., M2) in an acceptor human immunoglobulin molecule, and one or more human amino acid residues in the Fv framework region (FR), which are amino acid residues that flank the CDRs. Human framework region residues of the immunoglobulin can be replaced with corresponding non-human residues. Residues in the human framework regions can therefore be substituted with a corresponding residue from the non-human CDR donor antibody to alter, generally to improve, antigen affinity or specificity, for example. In addition, a humanized antibody may include residues, which are found neither in the human antibody nor in the donor CDR or framework sequences. For example, a framework substitution at a particular position that is not found in a human antibody or the donor non-human antibody may be predicted to improve binding affinity or specificity human antibody at that position. Antibody framework and CDR substitutions based upon molecular modeling are well known in the art, e.g., by modeling of the interactions of the CDR and framework residues to identify framework residues important for antigen binding and sequence comparison to identify unusual framework residues at particular positions (see, e.g., U.S. Pat. No. 5,585,089; and Riechmann et al., Nature 332:323 (1988)). Antibodies referred to as “primatized” in the art are within the meaning of “humanized” as used herein, except that the acceptor human immunoglobulin molecule and framework region amino acid residues may be any primate residue, in addition to any human residue.

As used herein, the term “chimeric” and grammatical variations thereof, when used in reference to an antibody, means that the amino acid sequence of the antibody contains one or more portions that are derived from, obtained or isolated from, or based upon two or more different species. That is, for example, a portion of the antibody may be human (e.g., a constant region) and another portion of the antibody may be non-human (e.g., a murine variable region). Thus, a chimeric antibody is a molecule in which different portions of the antibody are of different species origins. Unlike a humanized antibody, a chimeric antibody can have the different species sequences in any region of the antibody. An example of a chimeric antibody is antibody no. 2074, which has mouse lambda light chain and human gamma heavy chain.

As used herein, the terms “M2,” “M2 protein,” “M2 sequence” and “M2 domain” refer to all or a portion of an M2 protein sequence (e.g., a subsequence such as the extracellular domain) isolated from, based upon or present in any naturally occurring or artificially produced influenza virus strain or isolate. Thus, the term M2 and the like include naturally occurring M2 sequence variants produced by mutation during the virus life-cycle or produced in response to a selective pressure (e.g., drug therapy, expansion of host cell tropism or infectivity, etc.), as well as recombinantly or synthetically produced M2 sequences.

M2 antibodies of the invention include antibodies having kappa or lambda light chain sequences, either full length as in naturally occurring antibodies, mixtures thereof (i.e, fusions of kappa and lambda chain sequences), and subsequences thereof, as described in detail below. Naturally occurring antibody molecules contain two kappa and two lambda light chains. The primary difference between kappa and lambda light chains is in the sequences of the constant region.

Invention M2 antibodies include antibodies having the binding specificity of the M2 antibodies exemplified herein, e.g., having the binding specificity of an antibody denoted as no. 2074 (ATCC Deposit No. PTA-4025), 161(ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos., respectively; American Type Culture Collection, Manassas, Va., USA). In one aspect, an M2 antibody has a heavy (H) or light (L) chain sequence, or a subsequence thereof, as set forth in any of nos. 2074 (ATCC Deposit No. PTA-4025), 161 (ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40GI (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos., respectively; American Type Culture Collection, Manassas, Va., USA), provided that the heavy or light chain sequence, or subsequence of the antibody has the binding specificity of no. 2074 (ATCC Deposit No. PTA-4025), 161(ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos. , respectively; American Type Culture Collection, Manassas, Va., USA).

The term “binding specificity,” when used in reference to an antibody means that the antibody specifically binds to all or a part of the same antigenic epitope as the reference antibody. Thus, an M2 antibody having the binding specificity of the antibody denoted as no. 2074 specifically binds to all or a part of the same epitope as the M2 antibody denoted as no. 2074; an M2 antibody having the binding specificity of the antibody denoted as 161 specifically binds to all or a part of the same epitope as the M2 antibody denoted as 161; an M2 antibody having the binding specificity of the antibody denoted as N547 specifically binds to all or a part of the same epitope as the M2 antibody denoted as N547; an M2 antibody having the binding specificity of the antibody denoted as L66 specifically binds to all or a part of the same epitope as the M2 antibody denoted as L66; an M2 antibody having the binding specificity of the antibody denoted as C40G1 specifically binds to all or a part of the same epitope as the M2 antibody denoted as C40G1; and so on and so forth.

A part of an antigenic epitope means a subsequence or a portion of the epitope. For example, if an epitope includes 8 contiguous amino acids, a subsequence and, therefore, a part of an epitope may be 7 or fewer amino acids within this 8 amino acid sequence epitope. In addition, if an epitope includes non-contiguous amino acid sequences, such as a 5 amino acid sequence and an 8 amino acid sequence which are not contiguous with each other, but form an epitope due to protein folding, a subsequence and, therefore, a part of an epitope may be either the 5 amino acid sequence or the 8 amino acid sequence alone.

Antibodies having the binding specificity of the M2 antibodies exemplified herein compete with the binding of no. 2074 (ATCC Deposit No. PTA-4025), 161 (ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L409-S212, S80, S900 (ATCC Deposit Nos., respectively; American Type Culture Collection, Manassas, Va., USA). An antibody of the invention having binding specificity of the M2 antibodies exemplified herein may be characterized by any method known in the art for determining competitive binding, for example, the immunoassays disclosed herein. Because the binding affinity may differ from the exemplified antibodies, the antibodies will vary in their ability to compete for binding to M2. In particular embodiments, the antibody competitively inhibits binding by at least 95%, at least 90%, at least 85%, at least 80%, at least 75%, at least 70%, at least 65%, at least 60%, at least 55%, at least 50%, at least 45%, at least 40%, at least 35%, or at least 30%, or less.

Epitopes typically are short amino acid sequences, e.g. about five to 15 amino acids in length. Systematic techniques for identifying epitopes are known in the art and are described, for example, in U.S. Pat. No. 4,708,871. Briefly, a set of overlapping oligopeptides derived from an M2 antigen may be synthesized and bound to a solid phase array of pins, with a unique oligopeptide on each pin. The array of pins may comprise a 96-well microtiter plate, permitting one to assay all 96 oligopeptides simultaneously, e.g., for binding to an anti-M2 monoclonal antibody. Alternatively, phage display peptide library kits (New England BioLabs) are currently commercially available for epitope mapping. Using these methods, binding affinity for every possible subset of consecutive amino acids may be determined in order to identify the epitope that a particular antibody binds. Epitopes may also be identified by inference when epitope length peptide sequences are used to immunize animals from which antibodies that bind to the peptide sequence are obtained.

Invention M2 antibodies also include human, humanized and chimeric antibodies having the same binding affinity and having substantially the same binding affinity as the M2 antibodies exemplified herein. For example, an M2 antibody of the invention may have an affinity greater or less than 2-5, 5- 10, 10-100, 100-100 or 1000-10,000 fold affinity as the reference antibody. Thus, in additional embodiments the invention provides M2 antibodies having the same binding affinity and having substantially the same binding affinity as the antibodies denoted as no. 2074 (ATCC Deposit No. PTA-4025), 161(ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos. , respectively; American Type Culture Collection, Manassas, Va., USA), provided that the heavy or light chain sequence, or subsequence thereof has the binding specificity of no. 2074 (ATCC Deposit No. PTA-4025), 161(ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos. , respectively; American Type Culture Collection, Manassas, Va., USA).

As used herein, the term “the same,” when used in reference to antibody binding affinity, means that the dissociation constant (K_{D}) is within about 5 to 100 fold of the reference antibody (5-100 fold greater affinity or less affinity than the reference -antibody). The term “substantially the same” when used in reference to antibody binding affinity, means that the dissociation constant (K_{D}) is within about 5 to 5000 fold of the reference antibody (5-5000 fold greater affinity or less affinity than the reference antibody).

Additional antibodies included in the invention have a binding specificity of the antibodies denoted as no. 2074 (ATCC Deposit No. PTA-4025), 161 (ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos., respectively; American Type Culture Collection, Manassas, Va., USA), and binding affinity for M2 with a dissociation constant (Kd) less than 5×10⁻² M, 10⁻² M, 5×10⁻³ M, 10⁻³ M, 5×10⁻⁴ M, 10⁻⁴ M, 5×10⁻⁵ M, 10⁻⁵ M 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 5×10⁻¹¹ M, 10⁻¹² M, 5×10⁻¹² M, 10⁻¹³ M, 5×10⁻¹⁴ M, 10⁻¹⁴ M, 5×10⁻¹⁵ M, and 10⁻¹⁵ M.

Invention human M2 antibodies include antibodies having at least a part of one or more anti-influenza activities of the M2 antibodies exemplified herein (e.g., inhibit influenza virus infection of a cell in vitro or in vivo, inhibit influenza virus proliferation or replication, decrease one or more symptoms or complications associated with influenza virus infection, decrease susceptibility to influenza virus infection, etc.). Thus, in additional embodiments the invention provides M2 antibodies having at least a part of one or more anti-influenza activities of the antibodies denoted as no. 2074 (ATCC Deposit No. PTA-4025), 161(ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos., respectively; American Type Culture Collection, Manassas, Va., USA).

The term “activity,” when used in comparing an antibody to a reference antibody, means that the antibody has at least a part of an activity as the reference antibody, for example, binding affinity, binding specificity or anti-influenza activity. Thus, an antibody having an activity of the M2 antibody denoted as N547 has at least a part of one or more activities of the M2 antibody denoted as N547; an antibody having an activity of the M2 antibody denoted as L66 has at least a part of one or more activities of the M2 antibody denoted as L66; an antibody having an activity of the M2 antibody denoted as C40G 1 has at least a part of one or more activities of the M2 antibody denoted as C40G1; and so on and so forth. The term “at least a part” means that the antibody may have less activity but the antibody retains at least some of the activity of the reference M2 antibody, e.g., at least partial binding affinity for M2, at least partial anti-influenza activity, etc.

Antibodies having an activity of exemplified human M2 antibodies can be identified using binding assay with plate-bound M2 peptide as a coating antigen (ELISA), binding assay to M2 protein on viral infected MDCK cells (cell based ELISA), and specific inhibition of antibody binding to M2 on the viral infected MDCK cells with M2 peptide (M2 extracellular portion). Additional assays include in vitro cell infectivity assays with influenza virus (Zebedee et al. J. Virology 62:2762(1988)) as-well as in vivo animal assays as set forth in Examples 1, 3 and 4.

Methods of producing human antibodies are disclosed herein and known in the art. For example, as disclosed herein M2 protein conjugated to KLH or BSA was used to immunize human transchromosomic KM mice (WO 02/43478) or HAC mice (WO 02/092812). KM mice or HAC mice express human immunoglobulin genes. Using conventional hybridoma technology, splenocytes from immunized mice that were high responders to M2 antigen were isolated and fused with myeloma cells. Twelve monoclonal antibodies were obtained, denoted no. 2074, C40, L17, L30, L40, L66, N547, S212, S80, S900, F1, and F2, that reacted to M2 peptide and/or M2-BSA conjugates, but did not bind to the BSA or KLH carriers. An overview of the technology for producing human antibodies is described in Lonberg and Huszar, Int. Rev. Immunol. 13:65 (1995). Transgenic animals with one or more human immunoglobulin genes (kappa or lambda) that do not express endogenous immunoglobulins are described, for example in, U.S. Pat. No. 5,939,598. Human antibodies are also available from commercial vendors such as Abgenix. Inc. (Freemont, Calif.) and Genpharm (San Jose, Calif.). Additional methods for producing human antibodies and human monoclonal antibodies are described (see, e.g., WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598).

M2 Monoclonal antibodies can also be readily generated using other techniques including hybridoma, recombinant, and phage display technologies, or a combination thereof (see U.S. Pat. Nos. 4,902,614, 4,543,439, and 4,411,993; see, also Monoclonal Antibodies Hybridomas: A New Dimension in Biological Analyses, Plenum Press, Kennett, McKearn, and Bechtol (eds.), 1980, and Harlow et al., Antibodies: A Laboratory Manual, Cold Spring Harbor Laboratory Press, 2nd ed. 1988). Suitable techniques that additionally may be employed in the method including M2 affinity purification, non-denaturing gel purification, HPLC or RP-HPLC, purification on protein A column, or any combination of these techniques. The antibody isotype can be determined using an ELISA assay, for example, a human Ig can be identified using mouse Ig-absorbed anti-human Ig.

Antibodies can be humanized using a variety of techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l. Acad. Sci. USA 91:969 (1994)), and chain shuffling (U.S. Pat. No. 5,565,332). Human consensus sequences (Padlan Mol. Immunol. 31:169 (1994); and Padlan Mol. Immunol. 28:489 (1991)) have previously used to humanize antibodies (Carter et al. Proc. Natl. Acad. Sci. USA 89:4285 (1992); and Presta et al. J. Immunol. 151:2623 (1993)).

Methods for producing chimeric antibodies are known in the art (e.g., Morrison, Science 229:1202 (1985); Oi et al., BioTechniques 4:214 (1986); Gillies et al., (1989) J. Immunol. Methods 125:191; and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

M2 protein suitable for generating antibodies can be produced by any of a variety of standard protein purification or recombinant expression techniques known in the art. For example, M2 can be produced by standard peptide synthesis techniques, such as solid-phase synthesis. A portion of the protein may contain an amino acid sequence such as a T7 tag or polyhistidine sequence to facilitate purification of expressed or synthesized M2. M2 peptides may be expressed in a cell and protein produced by the cells may be purified. M2 protein may be expressed as a part of a larger protein by recombinant methods.

Forms of M2 suitable for generating an immune response include peptide subsequences of full length M2 (e.g., typically four to five amino acids or more in length). Additional forms of M2 include M2 containing preparations or extracts, partially purified M2 as well as cells or viruses that express M2 or preparations of such expressing cells or viruses.

Animals which may be immunized include mice, rabbits, rats, sheep, goats, or guinea pigs; such animals may be genetically modified to include human IgG gene loci. Additionally, to increase the immune response, M2 can be coupled to another protein such as ovalbumin or keyhole limpet hemocyanin (KLH), thyroglobulin and tetanus toxoid, or mixed with an adjuvant such as Freund's complete or incomplete adjuvant. Initial and any optional subsequent immunization may be through intraperitoneal, intramuscular, intraocular, or subcutaneous routes. Subsequent immunizations may be at the same or at different concentrations of M2 antigen preparation, and may be at regular or irregular intervals.

Thus, in another embodiment, the invention provides methods of producing human M2 antibodies, including antibodies having one or more an anti-influenza activities, such as inhibiting influenza virus infection, replication, proliferation, or titre, or inhibiting increases in virus replication, proliferation or titre, or reducing the severity or duration of one or more symptoms or complications associated with influenza infection, or susceptibility to infection, or having broad reactivity against various influenza virus strains or isolates. In one embodiment, a method includes administering M2 or an immunogenic fragment thereof to an animal (e.g., a mouse) capable of expressing human immunoglobulin; screening the animal for expression of human M2 antibody; selecting an animal that produces a human M2 antibody; isolating an antibody from the animal that produces human M2 antibody; and determining whether the human M2 antibody binds to M2. In another embodiment, a method includes administering human M2 or an immunogenic fragment thereof to an animal (e.g., a mouse) capable of expressing human immunoglobulin; isolating spleen cells from the mouse that produces human M2 antibody; fusing the spleen cells with a myeloma cell to produce a hybridoma; and screening the hybridoma for expression of a human M2 antibody that has an anti-influenza activity.

The invention further provides human M2 antibodies that have been modified. Examples of modifications include one or more amino acid substitutions, additions or deletions of the antibody, provided that the modified antibody has all or at least part of an activity of unmodified M2 antibody, e.g., an anti-influenza activity.

A particular example of a modification is where an antibody of the invention is altered to have a different isotype or subclass by, for example, substitution of the heavy chain constant region (see, for example, Example 2). An alteration of the Ig subclass of an M2 antibody C40 from IgG4 to IgG1 results in an improvement in an anti-influenza activity. Thus, modifications include deleting large regions of amino acid sequences from an invention antibody and substituting the region with another amino acid sequence, whether the sequence is greater or shorter in length than the deleted region.

Additional modifications of M2 antibodies included in the invention are antibody derivatives i.e., the covalent attachment of any type of molecule to the antibody. Specific examples of antibody derivatives include antibodies that have been glycosylated, acetylated, phosphorylated, amidated, formylated, ubiquitinated, and derivatization by protecting/blocking groups and any of numerous chemical modifications.

Individual amino acid substitutions may be with the same amino acid, except that a naturally occurring L-amino acid is substituted with a D-form amino acid. Amino acid substitutions can be conservative or non-conservative and may be in the constant or variable regions of the antibody. One or a few conservative amino acid substitutions in constant or variable regions are likely to be tolerated. Particular examples of conservative amino acid substitutions are Ile, Val, Leu or Ala for one another; Lys and Arg for one another; Glu and Asp for one another; and Gln and Asn for one another. Non-conservative substitution of multiple amino acids in hypervariable regions is likely to affect binding activity, specificity or antibody function or activity. Thus, substitutions in a hypervariable region may be assayed for their effect in order to identify those retaining at least a part of the binding activity, specificity or antibody function or activity of unsubstituted antibody. Such antibodies having amino acid substitutions are included so long as at least a part of binding specificity, binding affinity, or an anti-influenza activity of unmodified human M2 antibody is retained by the substituted antibody.

Human monoclonal M2 antibodies of the invention therefore include subsequences (e.g., fragments) and modified forms (e.g., sequence variants) as set forth herein. In particular embodiments, human M2 antibody subsequences include an Fab, Fab′ and F(ab)2, Fd, single-chain Fvs (scFv), single-chain antibodies, disulfide-linked Fvs (sdFv) and V_{L} or V_{H} domain fragments. In particular aspects, an Fab, Fab′ and F(ab′)2, Fd, single-chain Fvs (scFv), single-chain antibodies, disulfide-linked Fvs (sdFv) and V_{L} or V_{H} domain subsequence has the same binding affinity, substantially the same binding affinity, the same binding specificity, or one or more anti-influenza activities, e.g., efficacy in inhibiting influenza infection of a cell in vitro or in vivo as the reference M2 antibody (e.g., the full length or unmodified M2 antibody). M2-binding antibody subsequences, including single-chain antibodies, include variable region(s) alone or in combination with all or a portion of one or more of the following: hinge region, CH1, CH2, and CH3 domains. Also included are antigen-binding subsequences of any combination of variable region(s) with a hinge region, CH1, CH2, and CH3 domains.

M2 antibody subsequences (e.g., Fab, Fab′, F(ab′)2, Fd, scFv, sdFv and V_{L} or V_{H}) of the invention can be prepared by proteolytic hydrolysis of the antibody, for example, by pepsin or papain digestion of whole antibodies. The terms “functional subsequence” and “functional fragment” when referring to an antibody of the invention refers to a portion of an antibody that retains at least a part of one or more functions or activities as the intact reference antibody.

Antibody fragments can be produced by enzymatic cleavage with pepsin provide a 5S fragment denoted F(ab′)₂. This fragment can be further cleaved using a thiol reducing agent to produce 3.5S Fab′ monovalent fragments. Alternatively, an enzymatic cleavage using pepsin produces two monovalent Fab′ fragments and the Fe fragment directly (see, e.g., Goldenberg, U.S. Pat. Nos. 4,036,945 and 4,331,647; and Edelman et al. Methods in Enymology 1:422 (1967)). Other methods of cleaving antibodies, such as separation of heavy chains to form monovalent light-heavy chain fragments, further cleavage of fragments, or other enzymatic or chemical may also be used. Genetic techniques include expression of all or a part of the M2 antibody gene into a host cell such as Cos cells or E. coli. The recombinant host cells synthesize intact or single antibody chain, such as a scFv (see, e.g., Whitlow et al., In: Methods: A Companion to Methods in Enzymology 2:97 (1991), Bird et al., Science 242:423 (1988); and U.S. Pat. No. 4,946,778). Single-chain Fvs and antibodies can be produced as described in U.S. Pat. Nos. 4,946,778 and 5,258,498; Huston et al., Methods Enzymol. 203:46 (1991); Shu et al., Proc. Natl. Acad. Sci. USA 90:7995 (1993); and Skerra et al., Science 240:1038 (1988).

Another particular example of a modified M2 antibody having an amino acid addition is one in which a second heterologous sequence, i.e., heterologous functional domain is attached that confers a distinct or complementary function upon the antibody. For example, an amino acid tag such as T7 or polyhistidine can be attached to M2 antibody in order to facilitate purification or detection of M2 or influenza virus(es). Yet another example is an antiviral attached to an M2 antibody in order to target cells infected with influenza for virus killing, proliferation inhibition, replication inhibition, etc. Thus, in other embodiments the invention provides M2 antibodies and a heterologous domain, wherein the domain confers a distinct function, i.e. a heterologous functional domain, on the antibody.

Heterologous functional domains are not restricted to amino acid residues. Thus, a heterologous functional domain can consist of any of a variety of different types of small or large functional moieties. Such moieties include nucleic acid, peptide, carbohydrate, lipid or small organic compounds, such as a drug (e.g., an antiviral).

Linker sequences may be inserted between the antibody sequence and the heterologous functional domain so that the two entities maintain, at least in part, a distinct function or activity. Linker sequences may have one or more properties that include a flexible conformation, an inability to form an ordered secondary structure or a hydrophobic or charged character which could promote or interact with either domain. Amino acids typically found in flexible protein regions include Gly, Asn and Ser. Other near neutral amino acids, such as Thr and Ala, may also be used in the linker sequence. The length of the linker sequence may vary without significantly affecting a function or activity of the fusion protein (see, e.g., U.S. Pat. No. 6,087,329).

Additional examples of heterologous functional domains are detectable labels. Thus, in another embodiment, the invention provides human M2 antibodies that are detectably labeled.

Specific examples of detectable labels include fluorophores, chromophores, radioactive isotopes (e.g., S³⁵, P³², I¹²⁵ ), electron-dense reagents, enzymes, ligands and receptors. Enzymes are typically detected by their activity. For example, horseradish peroxidase is usually detected by its ability to convert a substrate such as 3,3′,5,5′-tetramethylbenzidine (TMB) to a blue pigment, which can be quantified. Ligands may bind other molecules such as biotin, which may bind avidin or streptavidin, and IgG, which can bind protein A.

It is understood that a M2 antibody may have two or more variations, modifications or labels. For example, a monoclonal antibody may be coupled to biotin to detect its presence with avidin as well as labeled with I¹²⁵ so that it provides a detectable signal. Other permutations and possibilities will be readily apparent to those of ordinary skill in the art, and are considered to be within the scope of the invention.

The invention further provides nucleic acids encoding the human M2 antibodies of the invention, including modified forms, fragments, chimeras, etc. In particular embodiments, a nucleic acid encodes intact or single chain M2 antibody denoted as no. 2074 (ATCC Deposit No. PTA-4025), 161(ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos., respectively; American Type Culture Collection, Manassas, Va., USA).

The terms “nucleic acid” or “polynucleotide” are used interchangeably to refer to all forms of nucleic acid, including deoxyribonucleic acid (DNA) and ribonucleic acid (RNA). The nucleic acids can be double, single strand, or triplex, linear or circular. Nucleic acids include genomic DNA, cDNA, and antisense. RNA nucleic acid can be spliced or unspliced mRNA, rRNA, tRNA or antisense. Nucleic acids of the invention include naturally occurring, synthetic, as well as nucleotide analogues and derivatives. Such altered or modified polynucleotides include analogues that provide nuclease resistance, for example.

Nucleic acid can be of any length. For example, a subsequence of any of no. 2074 (ATCC Deposit No. PTA-4025), 161(ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos. , respectively; American Type Culture Collection, Manassas, Va., USA) that encodes a proteins having one or more anti-influenza activities. In a particular embodiment, a nucleic acid includes a heavy-chain variable sequence and light-chain variable sequence as set forth in SEQ ID NO: 9 and SEQ ID NO: 10. In another particular embodiment, a nucleic acid encodes a heavy-chain variable sequence and light-chain variable sequence as set forth in the SEQ ID NO: 11 and SEQ ID NO: 12.

As a result of the degeneracy of the genetic code, nucleic acids include sequences that are degenerate with respect to sequences encoding no. 2074 (ATCC Deposit No. PTA-4025), 161(ATCC Deposit No. PTA-4026;), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos., respectively; American Type Culture Collection, Manassas, Va., USA) subsequences thereof and modified forms as set forth herein.

Nucleic acid can be produced using any of a variety of well known standard cloning and chemical synthesis methods and can be altered intentionally by site-directed mutagenesis or other recombinant techniques known to those skilled in the art. Purity of polynucleotides can be determined through sequencing, gel electrophoresis and the like.

Nucleic acids of the invention may be inserted into a nucleic acid construct in which expression of the nucleic acid is influenced or regulated by an “expression control element,” referred to herein as an “expression cassette.” The term “expression control element” refers to one or more nucleic acid sequence elements that regulate or influence expression of a nucleic acid sequence to which it is operatively linked. An expression control element can include, as appropriate, promoters, enhancers, transcription terminators, gene silencers, a start codon (e.g., ATG) in front of a protein-encoding gene, etc.

An expression control element operatively linked to a nucleic acid sequence controls transcription and, as appropriate, translation of the nucleic acid sequence. The term “operatively linked” refers to a juxtaposition wherein the referenced components are in a relationship permitting them to function in their intended manner. Typically expression control elements are juxtaposed at the 5′ or the 3′ ends of the genes but can also be intronic.

Expression control elements include elements that activate transcription constitutively, that are inducible (i.e., require an external signal for activation), or derepressible (i.e., require a signal to turn transcription off; when the signal is no longer present, transcription is activated or “derepressed”). Also included in the expression cassettes of the invention are control elements sufficient to render gene expression controllable for specific cell-types or tissues (i.e., tissue-specific control elements). Typically, such elements are located upstream or downstream (i.e., 5′ and 3′) of the coding sequence. Promoters are generally positioned 5′ of the coding sequence. Promoters, produced by recombinant DNA or synthetic techniques, can be used to provide for transcription of the polynucleotides of the invention. A “promoter” is meant a minimal sequence element sufficient to direct transcription.

The nucleic acids of the invention may be inserted into a plasmid for propagation into a host cell and for subsequent genetic manipulation if desired. A plasmid is a nucleic acid that can be stably propagated in a host cell, plasmids may optionally contain expression control elements in order to drive expression of the nucleic acid encoding M2 antibody in the host cell. A vector is used herein synonymously with a plasmid and may also include an expression control element for expression in a host cell. Plasmids and vectors generally contain at least an origin of replication for propagation in a cell and a promoter. Plasmids and vectors are therefore useful for genetic manipulation of M2 antibody encoding nucleic acids, producing M2 antibodies or antisense, and expressing the M2 antibodies in host cells or organisms, for example.

Nucleic acids encoding variable regions of the antibody heavy and light chains, or encoding full length antibody heavy and light chains can be isolated from a hybridoma. Isolated nucleic acids may be inserted into a suitable expression vector, and introduced into suitable host cells such as yeast or CHO cells which can be cultured for the production of recombinant M2 antibodies.

Bacterial system promoters include T7 and inducible promoters such as pL of bacteriophage λ, plac, ptrp, ptac (ptrp-lac hybrid promoter) and tetracycline responsive promoters. Insect cell system promoters include constitutive or inducible promoters (e.g., ecdysone). Mammalian cell constitutive promoters include SV40, RSV, bovine papilloma virus (BPV) and other virus promoters, or inducible promoters derived from the genome of mammalian cells (e.g., metallothionein IIA promoter; heat shock promoter) or from mammalian viruses (e.g., the adenovirus late promoter; the inducible mouse mammary tumor virus long terminal repeat). Alternatively, a retroviral genome can be genetically modified for introducing and directing expression of a M2 antibody in appropriate host cells.

Expression systems further include vectors designed for in vivo use. Particular non-limiting examples include adenoviral vectors (U.S. Pat. Nos. 5,700,470 and 5,731,172), adeno-associated vectors (U.S. Pat. No. 5,604,090), herpes simplex virus vectors (U.S. Pat. No. 5,501,979), retroviral vectors (U.S. Pat. Nos. 5,624,820, 5,693,508 and 5,674,703), BPV vectors (U.S. Pat. No. 5,719,054) and CMV vectors (U.S. Pat. No. 5,561,063).

Yeast vectors include constitutive and inducible promoters (see, e.g., Ausubel et al., In: Current Protocols in Molecular Biology, Vol. 2, Ch. 13, ed., Greene Publish. Assoc. & Wiley Interscience, 1988; Grant et al. Methods in Enzymology, 153:516 (1987), eds. Wu & Grossman; Bitter Methods in Enzymology, 152:673 (1987), eds. Berger & Kimmel, Acad. Press, N.Y.; and, Strathem et al., The Molecular Biology of the Yeast Saccharomyces (1982) eds. Cold Spring Harbor Press, Vols. I and II). A constitutive yeast promoter such as ADH or LEU2 or an inducible promoter such as GAL may be used (R. Rothstein In: DNA Cloning, A Practical Approach, Vol. 11, Ch. 3, ed. D. M. Glover, IRL Press, Wash., D.C., 1986). Vectors that facilitate integration of foreign nucleic acid sequences into a yeast chromosome, via homologous recombination for example, are known in the art. Yeast artificial chromosomes (YAC) are typically used when the inserted polynucleotides are too large for more conventional vectors (e.g., greater than about 12 kb).

Host cells including nucleic acids encoding human M2 antibodies are also provided. In one embodiment, the host cell is a prokaryotic cell. In another embodiment, the host cell is a eukaryotic cell. In various aspects, the eukaryotic cell is a yeast or mammalian (e.g., human, primate, etc.) cell.

As used herein, a “host cell” is a cell into which a nucleic acid is introduced that can be propagated, transcribed, or encoded M2 antibodyexpressed. The term also includes any progeny or subclones of the host cell. Progeny cells and subclones need not be identical to the parental cell since there may be mutations that occur during replication and proliferation. Nevertheless, such cells are considered to be host cells of the invention.

Host cells include but are not limited to microorganisms such as bacteria and yeast; and plant, insect and mammalian cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for stable expression, are provided.

Expression vectors also can contain a selectable marker conferring resistance to a selective pressure or identifiable marker (e.g., β-galactosidase), thereby allowing cells having the vector to be selected for, grown and expanded. Alternatively, a selectable marker can be on a second vector which is cotransfected into a host cell with a first vector containing an invention polynucleotide.

Selection systems include but are not limited to herpes simplex virus thymidine kinase gene (Wigler et al., Cell 11:223 (1977)), hypoxanthine-guanine phosphoribosyltransferase gene (Szybalska et al., Proc. Natl. Acad. Sci. USA 48:2026 (1962)), and adenine phosphoribosyltransferase (Lowy et al., Cell 22:817 (1980)) genes which can be employed in tk-, hgprt- or aprt-cells respectively. Additionally, antimetabolite resistance can be used as the basis of selection for dhfr, which confers resistance to methotrexate (O'Hare et al., Proc. Natl. Acad. Sci. USA 78:1527 (1981)); the gpt gene, which confers resistance to mycophenolic acid (Mulligan et al., Proc. Natl. Acad. Sci. USA 78:2072 (1981)); neomycin gene, which confers resistance to aminoglycoside G-418 (Colberre-Garapin et al., J. Mol. Biol. 150:1(1981)); puromycin; and hygromycin gene, which confers resistance to hygromycin (Santerre et al., Gene 30:147 (1984)). Additional selectable genes include trpB, which allows cells to utilize indole in place of tryptophan; hisD, which allows cells to utilize histinol in place of histidine (Hartman et al., Proc. Natl. Acad. Sci. USA 85:8047 (1988)); and ODC (omithine decarboxylase), which confers resistance to the omithine decarboxylase inhibitor, 2-(difluoromethyl)-DL-ornithine, DFMO (McConlogue (1987) In: Current Communications in Molecular Biology, Cold Spring Harbor Laboratory).

Methods for treating influenza virus infection of a subject include administering to the subject an amount of a human, humanized or chimeric antibody that specifically binds influenza M2 protein effective to treat influenza virus infection of the subject. The antibody can be administered prior to infection, i.e., prophylaxis, substantially contemporaneously with infection, or following infection of the subject, i.e., therapeutic treatment.

Methods of the invention include providing a therapeutic benefit to a subject, for example, reducing or decreasing one or more symptoms or complications associated with influenza virus infection, reducing or inhibiting increases in virus titer, virus replication, virus proliferation, or an amount of a viral protein of one or more influenza virus strains or isolates. Symptoms or complications associated with influenza virus infection that can be reduced or decreased include, for example, chills, fever, cough, sore throat, nasal congestion, sinus congestion, nasal infection, sinus infection, body ache, head ache, fatigue, pneumonia, bronchitis, ear infection or ear ache. A therapeutic benefit can also include reducing susceptibility of a subject to influenza virus infection or hastening a subject's recovery from influenza virus infection.

In one embodiment, a method includes administering to the subject an amount of a human, humanized or chimeric antibody that specifically binds influenza virus M2 effective to inhibit virus infection of the subject or reduce susceptibility of the subject to virus infection by one or more influenza virus strains or isolates. In various aspects, the antibody is administered prior to (prophylaxis), substantially contemporaneously with or following infection of the subject (therapeutic). The antibody can provide a therapeutic benefit which includes, for example, reducing or decreasing the severity or duration of one or more symptoms or complications of influenza virus infection (e.g., chills, fever, cough, sore throat, nasal congestion, sinus congestion, nasal infection, sinus infection, body ache, head ache, fatigue, pneumonia, bronchitis, ear infection or ear ache), virus titer or an amount of a viral protein of one or more influenza virus strains or isolates, or susceptibility of a subject to infection by one or more influenza virus strains or isolates.

Therapeutic benefits and therefore methods for preventing or inhibiting an increase in influenza virus titer, virus replication, virus proliferation or an amount of an influenza viral protein in a subject are further provided. In one embodiment, a method includes administering to the subject an amount of a human, humanized or chimeric antibody that specifically binds influenza M2 effective to prevent an increase in influenza virus titer, virus replication or an amount of an influenza viral protein of one or more influenza strains or isolates in the subject.

Methods for protecting a subject from infection, decreasing susceptibility of a subject to infection and hastening a subject's recovery from infection by one or more influenza strains or isolates are additionally provided. In one embodiment, a method includes administering to the subject an amount of a human, humanized or chimeric antibody that specifically binds influenza M2 effective to protect the subject from virus infection, effective to decrease susceptibility of the subject to virus infection or hastening a subject's recovery from virus infection, by one or more influenza virus strains or isolates.

Methods of the invention can be practiced with any antibody having the binding specificity or the same or substantially the same binding affinity of an antibody produced by a cell line (e.g., a hybridoma or a CHO cell line) denoted as no. 2074 (ATCC Deposit No. PTA-4025; American Type Culture Collection, Manassas, Va., USA), 161 (ATCC Deposit No. PTA-4026; American Type Culture Collection, Manassas, Va., USA), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), L17 (ATCC Deposit No.; American Type Culture Collection, Manassas, Va., USA), and C40, L30, L40, S212, S80, S900 (ATCC Deposit Nos. , respectively; American Type Culture Collection, Manassas, Va., USA).

Methods of the invention, including therapeutic, diagnostic and purification/isolation methods are applicable to any influenza strain/isolate or combination of strains/isolates. Particular non-limiting examples of influenza strains are A/PR/8/34 or A/HK/8/68, or other strains selected from H1N1, H2N2, H3N2, H5N1, H9N2, H2N1, H4N6, H6N2, H7N2, H7N3, H4N8, H5N2, H2N3, H11N9, H3N8, H1N2, H11N2, H11N9, H7N7, H2N3, H6N1, H13N6, H7N1, H11N1, H7N2 and H5N3.

Human, humanized and chimeric M2 antibodies of the invention may be used alone or in combination with therapeutic agents having anti-influenza activity, e.g., that inhibit influenza virus infection, replication, proliferation, or reduce the severity or duration of one or more symptoms or complications associated with influenza virus infection. Examples of such combinations include pooled monoclonal antibodies containing two or more different M2 antibodies with different binding specificity, binding affinity, or efficacy in inhibiting influenza virus infection of a cell in vitro or in vivo. Accordingly, combination compositions including M2 antibodies are provided, as well as methods of using such combinations in accordance with the methods of the invention.

The methods of the invention, including treating influenza or a disorder or complication associated with influenza virus infection, likely results in an improvement in the subjects' condition, a reduction of the severity or duration of one or more symptoms or complications associated with influenza virus infection, or decreasing the subject's risk for developing symptoms or contracting the infection, e.g, susceptibility to influenza virus infection. An improvement therefore includes one or more decreased or reduced virus proliferation, replication, or titre, or symptoms or complications associated with influenza virus infection. An improvement also includes reducing the dosage frequency or amount of an antiviral drug or other agent used for treating a subject having or at risk of having an influenza virus infection, or a symptom or complication associated with influenza virus infection.

An improvement need not be complete ablation of any or all symptoms or complications associated with influenza virus infection. Rather, treatment may be any measurable or detectable anti-influenza virus effect or improvement as set forth herein. Thus, a satisfactory clinical endpoint is achieved when there is an incremental improvement or a partial reduction in the subjects condition or associated symptoms or complications, or an inhibition of worsening of the condition, over a short or long duration.

Subjects appropriate for treatment include those having or at risk of having influenza virus infection. Target subjects also include those at risk of developing an influenza associated symptom or complication. The invention methods are therefore applicable to treating a subject who is at risk of influenza virus infection or a complication associated with influenza virus infection. Prophylactic methods are therefore included.

At risk subjects appropriate for treatment include subjects exposed to other subjects having influenza virus, or where the risk of influenza virus infection is increased due to changes in virus infectivity or cell tropism, immunological susceptibility (e.g., an immunocompromised subject), or environmental factors.

M2 antibodies can be administered as a single or multiple dose e.g., one time per week for between-about 1 to 10 weeks, or for as long as appropriate, for example, to achieve a reduction in the severity of one or more symptoms or complications associated with influenza virus infection. Doses can vary depending upon whether the treatment is prophylactic or therapeutic, the severity of the associated disorder or complication being treated, the clinical endpoint desired, previous or simultaneous treatments, the general health, age, sex or race of the subject and other factors that will be appreciated by the skilled artisan. The skilled artisan will appreciate the factors that may influence the dosage and timing required to provide an amount sufficient for therapeutic benefit. Doses can be empirically determined or determined using animal disease models or optionally in human clinical trials.

The term “subject” refers to animals, typically mammalian animals, such as a non human primate (apes, gibbons, chimpanzees, orangutans, macaques), a domestic animal (dogs and cats), a farm animal (horses, cows, goats, sheep, pigs), experimental animal (mouse, rat, rabbit, guinea pig) and humans. Subjects include animal disease models, for example, the mouse animla model of influenza infection exemplified herein.

M2 antibodies of the invention, including modified forms, variants and subsequences thereof, and nucleic acids encoding M2 antibodies, can be incorporated into pharmaceutical compositions. Such pharmaceutical compositions are useful for administration to a subject in vivo or ex vivo.

Antibodies can be included in a pharmaceutically acceptable carrier or excipient prior to administration to a subject. As used herein the term “pharmaceutically acceptable” and “physiologically acceptable” includes solvents (aqueous or non-aqueous), solutions, emulsions, dispersion media, coatings, isotonic and absorption promoting or delaying agents, compatible with pharmaceutical administration. Such formulations can be contained in a tablet (coated or uncoated), capsule (hard or soft), microbead, emulsion, powder, granule, crystal, suspension, syrup or elixir. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. For transdermal administration, the active compounds are formulated into aerosols, sprays, ointments, salves, gels, or creams as generally known in the art.

Pharmaceutical formulations and delivery systems appropriate for the compositions and methods of the invention are known in the art (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky et al., Drug Delivery Systems, R. L. Juliano, ed., Oxford, N.Y. (1980), pp. 253-315)

The pharmaceutical formulations can be packaged in unit dosage form for ease of administration and uniformity of dosage. Unit dosage form as used herein refers to physically discrete units suited as unitary dosages for the subject to be treated; each unit containing a predetermined quantity of active compound calculated to produce a desired therapeutic effect in association with the pharmaceutical carrier or excipient.

The invention provides kits comprising M2 antibodies, nucleic acids encoding M2 antibodies and pharmaceutical formulations thereof, packaged into suitable packaging material. A kit typically includes a label or packaging insert including a description of the components or instructions for use in vitro, in vivo, or ex vivo, of the components therein. A kit can contain a collection of such components, e.g., two or more human M2 antibodies alone or in combination with an antiviral agent or drug.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions.

Kits of the invention therefore can additionally include labels or instructions for using the kit components in a method of the invention. Instructions can include instructions for practicing any of the methods of the invention described herein including treatment, detection, monitoring or diagnostic methods. Thus, for example, a kit can include a human M2 antibody that has one or more anti-influenza activities as set forth herein, together with instructions for administering the antibody in a treatment method of the invention.

The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include a growth medium (e.g., for an M2 antibody producing cell line), buffering agent, or a preservative or a stabilizing agent in a pharmaceutical formulation containing a human M2 antibody. Each component of the kit can be enclosed within an individual container and all of the various containers can be within a single package. Invention kits can be designed for cold storage. Invention kits can further be designed to contain human M2 antibody producing hybridoma or other host cells (e.g., CHO cells). The cells in the kit can be maintained under appropriate storage conditions until the cells are ready to be used. For example, a kit including one or more hybridoma or other cells can contain appropriate cell storage medium (e.g., 10-20% DMSO in tissue culture growth medium such as DMEM, α-MEM, etc.) so that the cells can be thawed and grown.

Human M2 antibodies of the invention are useful for isolating, detecting or purifying M2 polypeptides. Such methods include contacting a sample suspected of containing M2 (in solution, in solid phase, in vitro or in vivo, or in an intact cell or organism) with an M2 antibody under conditions allowing binding, and detecting the presence of M2, or purifying the bound M2 protein.

The invention therefore also provides methods for detecting M2 or influenza virus in a test sample. In one embodiment, a method includes contacting a sample having or suspected of having M2 or influenza virus with a human M2 antibody under conditions allowing detection of M2 in the sample and determining whether M2 is present in the test sample. Detection of M2 or influenza virus can be performed by conventional methods such as immunoprecipitation, western blotting, immunohistochemical staining or flow cytometry.

M2 and influenza virus detection methods are useful in diagnostic protocols for detecting M2 and influenza virus. For example, where increased or decreased levels of influenza virus are associated with development or regression of influenza infection, invention antibodies can be used to detect any increase or decrease in M2 or influenza virus. In addition, where it is desired to monitor levels of M2 or influenza virus following a treatment therapy that decreases M2 or influenza virus levels, invention antibodies can be used to detect such an increase or decrease in M2 or influenza virus levels before, during or following the treatment, over a long or short period of time.

The invention therefore also provides methods for detecting the presence of M2 or influenza virus in a test sample of a subject (containing biological fluid, cells, or a tissue or organ sample such as a biopsy). In one embodiment, a method includes contacting a sample having or suspected of having M2 or influenza virus obtained from a subject with a human M2 or influenza virus antibody under conditions allowing detection of M2 or influenza virus and determining whether M2 or influenza virus is present in the test sample from the subject.

Human M2 antibodies may also be utilized to monitor the presence of M2 or influenza virus for diagnosis or following treatment of a subject, or to measure in vivo levels of M2 in subjects. For example, sputum suspected of containing M2 or influenza virusis incubated with an M2 antibody, as described above, under conditions allowing binding to occur, detects the presence of M2 or influenza virus

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “an M2 antibody” includes a plurality of such antibodies and reference to “an anti influenza activity or function” can include reference to one or more activities or functions, and so forth.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example describes various materials and methods.

Peptide synthesis and peptide-KLH conjugates: M2 peptides were synthesized by Multiple Peptide Systems (San Diego, Calif.). Peptide purity was >95% after HPLC. The M2 peptide was then conjugated to KLH (M2-KLH) and BSA (M2-BSA) by the same company. The sequence of the extracellular 23-amino-acid M2 peptide is: SLLTEVETPIRNEWGCRCNDSSD (SEQ ID NO: 1).

Mice: Human trans-chromosomic mice (Ishida and Lonberg, IBC's 11^{th} Antibody Engineering Meeting. Abstract (2000); and Kataoka, S. IBC's 13^{th} Antibody Engineering Meeting. Abstract (2002)) harboring human chromosome fragments containing the human immunoglobulin region were obtained from Kirin Brewery Co., Ltd. (Japan) and were housed in the animal facility at the La Jolla Institute for Allergy and Immunology. C57BL/6J mice were purchased from Jackson Laboratories at Bar Harbor, Me. and were housed in the animal facility at the La Jolla Institute for Allergy and Immunology.

Immunization: M2-KLH or M2-BSA in PBS (GIBCO BRL, Rockville, Md.) was mixed with an equal volume of complete Freund's adjuvant (CFA) (Sigma, St. Louis, Mo.) and an emulsion was prepared. Mice were immunized with 20 μg of M2-KLH or M2-BSA in CFA subcutaneously and were boosted either subcutaneously with 20 μg of M2-KLH or M2-BSA in incomplete Freund's adjuvant (IFA) (Sigma, St. Louis, Mo.) or intraperitoneal injection with RIBI (Corixa, Hamilton Mont.) after 21 days and repeated once more following another 21 days. A final intraperitoneal and intravenous injection of 10 μg of M2 peptide without adjuvant was given 3 days before fusion.

ELISA: Antibody titers and antibody specificity as well as antibody production by hybridomas were determined by ELISA. In brief, 50 μl of M2-BSA or M2 peptide were coated on a 96-well flat bottom plate (Nunc, Denmark) at a concentration of 1 μg/ml with carbonate buffer (pH 9.6) overnight at 4° C. or at 37° C. for 1 hr. After washing twice with PBS/0.1% Tween 20, plates were blocked with PBS/1% BSA (Sigma, St. Louis, Mo.) at 37° C. for 30 min., the antibody or serum was added to the wells and the plates were incubated at 37° C. for 1 hr. After washing four times, diluted HRP conjugated goat anti-human Immunoglobulin gamma chain specific antibody (Jackson Immunoresearch Laboratory, West Grove, Pa.) was added to the wells and incubated for 1 hr at 37° C. After washing four times, TMB substrate solution (DAKO, CA) was added and incubated for 30 min at room temperature. The optical density at 450 nm was measured by a microplate reader.

Isotype ELISA: The isotype of the antibody produced by the hybridomas was determined by ELISA. In brief, 50 μl of M2-BSA or M2 peptide were coated on a 96-well flat bottom plate (Nunc, Denmark) at a concentration of 1 μg/ml with carbonate buffer (pH 9.6) overnight at 4° C. or at 37° C. for 1 hr. After washing twice with PBS/0.1% Tween 20, plates were blocked with PBS/1% BSA (Sigma, St. Louis, Mo.) at room temperature for 1 hr, the antibody was added to the wells and the plates were incubated at room temperature for 1 hr. After washing three times, either of diluted HRP-conjugated mouse anti-human IgG1, IgG2, IgG3 and IgG4 heavy chain detection antibodies (Zymed, San Francisco, Calif.) was added to the wells and incubated for 1 hr at room temperature. After washing three times, TMB substrate solution (DAKO, CA) was added and incubated for 30 min at room temperature. The optical density at 450 nm was measured by a microplate reader.

Influenza A virus-infected cell-based ELISA: MDCK cells (Madin-Darby Canine Kidney epithelial cells; ATCC, Rockville, Md.) were plated in a 96-well flat bottom plate (Falcon®) at 1.5×10⁵ cells per mL and 150 μl per well and cultured for 48 hr at 7%CO₂. After 48 hr the plate was washed twice with PBS and infected at room temperature for 30 minutes with 30 μl of 100-fold TCID₅₀ influenza A virus (A/PR/8/34 or A/HK/8/68; ATCC, Rockville, Md.) with periodically swirling. After infection, the plate was washed once with PBS and 150 μl of 1 μg/mL trypsin (TPCK-treated, Worthington, Biochem. Corp.) in Minimal Essential Media (Invitrogen Corp, CA) was added and the plate incubated for 27 hr. After infection, the cell monolayer was washed with PBS/1%FCS (GIBCO BRL, Rockville, Md.) three times and blocked with PBS/1%BSA/5%FCS at room temperature for 30 min. The antibodies were diluted and 50 μl added to each well and incubated at room temperature for 45 min. After washing 4 times, the HRP conjugated Rabbit anti-human immunoglobulin gamma chain antibody (DAKO, Denmark) was diluted 1:3000 and 50 μl added to each well and the plate was incubated at room temperature for 30 min. After washing 5 times, 100 μl of TMB substrate (DAKO, Denmark) containing 1 mM Levamisole solution (Vector Laboratories Inc. Burlingame, Calif.) was added and the plates were incubated at room temperature for 15 min. 50 μl of supernatant were transferred to a new 96-well plate (Nunc, Denmark) containing 100 μl stop solution (1N H₂SO₄) and the optical density (OD) at 450 nm was measured by a microplate reader. EC₅₀ of each antibody was calculated as previously described (Sette et al. Nature 328:395 (1987)). The OD data of no. 2074 antibody at 10 μg/ml was set as 100% as an internal control.

Peptide competition in Influenza A virus-infected cell-based ELISA: Virus-infected MDCK cells were prepared as described above. The M2 peptide and the anti M2 antibodies were mixed and incubated at room temperature for 30 min. After incubation,50 μl of the mixture of peptide and antibodies were added to blocked cells and incubated at room temperature for 30 minutes After washing 4 times, the HRP conjugated Rabbit anti-human immunoglobulin gamma chain antibody (DAKO, Denmark) was diluted 1:3000 and 50 μl added to each well and the plate was incubated at room temperature for 30 min. After washing for 5 times, 100 μl of TMB substrate (DAKO, Denmark) containing 1 mM Levamisole solution (Vector Laboratories Inc. Burlingame, Calif.) was added and the plates were incubated at room temperature for 15 min. Fifty μl of supernatant was transferred to a new 96-well plate (Nunc, Denmark) containing 100 μl stop solution (1N H₂SO₄) and the optical density at 450 nm was measured by a microplate reader.

Hybridoma production. The mouse having the highest antibody titer was selected for production of monoclonal antibodies. The spleen was harvested and single cell suspension was fused to a myeloma cell line (SP2/O-Ag14) (ATCC, Rockville, Md.) at a 3:1 ratio with 50% PEG (Boehringer Mannheim, Indianapolis, Ind.). The fusions were plated onto 96-well plate at an optimal density and cultured in complete RPMI-10 medium (RPMI 1640 with 10% FCS, 1% nonessential amino acids, 2 mM L-glutamine, 50 μM 2-ME, 100 U/ml penicillin and 100 μg/ml streptomycin sulfate) in a 5% CO₂, 37° C. incubator. Approximately 2000 hybridoma growing wells of each fusion were screened by ELISA. Cells positive for binding to the M2 peptide were transferred to 24 well plates and 4 rounds of limiting dilutions were performed to obtain monoclonal antibodies. Anti-M2 monoclonal antibodies were further confirmed by an Influenza A virus infected cell based ELISA.

Antibody purification: For antibody purification, hybridomas were cultured in an Integra system (INTEGRA Bioscience,Inc. Ijamsville, Md.) with hybridoma-SFM(GIBCO BRL, Rockville, Md.). Human monoclonal antibodies were purified from culture media using Protein A-Sepharose Fast Flow gel (Amersham Pharmacia Cat#17-0618-02, Uppsala, Sweden). Briefly, conditioned medium, containing an appropriate amount of the antibody for the column capacity, was filtered with a 0.22 μm disk filter (Minisarto-plus, Sartorius Cat#17822, Gettingen, Germany) and loaded onto a 2.0 ml Protein A-Sepharose Fast Flow column equilibrated with phosphate buffered saline (PBS). The column was washed with more than 40 ml of PBS and the antibody was eluted with 0.1 M Gly-HCl, pH3.6, 0.15 M NaCl. After the initial 1.0 ml of the elution buffer had passed through, 3 separate elution fractions were collected at a volume of 5.0 ml/ tube, and neutralized immediately with 250 μl of 1 M Tris-HCl, pH8.0. This purification procedure was repeated until all conditioned media were processed. Antibody concentration was determined with a human IgG-specific ELISA and all fractions containing the antibody were pooled and concentrated with a centrifugal concentrator (Vivaspin 20, 30,000 MWCO: Sartorius Cat#VS2022, Gettingen, Germany).

In order to remove pyrogen, the concentrated sample was buffer-exchanged into 20 mM sodium phosphate, pH6.6, and loaded onto a 0.5 ml SP-Sepharose HP column (Amersham Pharmacia, Cat#17-1087-01, Uppsala, Sweden) equilibrated with the same buffer. The pyrogen was removed by first passing the sample through a 2 ml Q-Sepharose Fast Flow column (Amersham Pharmacia, Cat#17-0510-01, Uppsala, Sweden) that was connected in series to a SP-Sepharose HP column. After application, the Q-Sepharose Fast Flow column was removed and the antibody was eluted with a linear gradient ranging from 0 to 0.5 M sodium chloride. The antibody was detected at 280 nm and the antibody containing fractions pooled. The sample was concentrated with a centrifugal concentrator and buffer-exchanged into PBS by using NAP25 desalting columns (Amersham Pharmacia, Cat#17-0852-02, Uppsala, Sweden). Antibody concentration was quantitated by a human IgG specific ELISA. Pyrogen levels of samples were determined to be less than 0.13 EU/mg of protein according to a Limulus Amebocyte Lysate (LAL) assay (Associates of Cape Cod, Inc., Falmouth, Mass.).

Isolation of Human Anti-M2 Antibody (C40) Genes:

Cultured hybridoma cells (113C-40-H-22), which produce C40 antibody (isotype: IgG4) were collected by centrifugation. 240 microgram of total RNA was purified from these cells using ISOGEN (NIPPONGENE, Co., Ltd.), and subsequently 3 microgram of polyA⁺ RNA was purified from 120 microgram of total RNA using OligotexTM-dT30<Super> (Takara Shuzo, CO., Ltd., Japan). SMART RACE cDNA Amplification Kit (Clontech, Co., Ltd., CA) was used for cloning of cDNA of variable region of immunoglobulin genes from polyA⁺ RNA of hybridoma cells as a source. Briefly, first strand cDNA was prepared by reverse transcriptase from 2 microgram of polyA⁺ RNA. This cDNA was used as a template for polymerase chain reaction (PCR) to amplify variable regions of both heavy chain and light chain which included leader sequences (HV and LV, respectively). The reaction was as follows: 2.5 U TaKaRa LA TaqTM DNA polymerase (Takara Shuzo, Co.,); 0.2 μM Primer for one side (for Heavy chain: IgG1p, for Light chain: hk-2, see Table 1); 0.2 μM Primer for the other side (UMP primer attached to SMART RACE Kit); 400 μM each dNTP mix; LA PCR Buffer II (Mg2+ plus) (final concentration is 1×); and cDNA template.

The thermocycling program was 94° C. for 5 min, and then 30 cycles at 94° C. for 10 sec and 68° C. for 1 min with an extension at 72° C. for 7 min. Amplified DNA fragments were collected after ethanol precipitation and subsequent agarose gel electrophoresis, and purified by QIAquick Gel Extraction Kit (Qiagen Co., Ltd., Germany). Nucleotide sequences of both PCR-amplified products (HV and LV) were confirmed with specific primers (HV: hh-4, LV: hk-5 and hk-6, see Table 1 for sequences of primers). Purified DNA fragments of HV and LV was integrated into pGEM®-T Easy Vector System (Promega Co.), and each construct plasmid was electroporated in E.coli, and then cloned. Nucleotide sequences of each insert (HV and LV) in construct plasmids were analysed using specific primers (SP6 and T7, see Table 1). Nucleotide sequences of both HV and LV from construct plasmids were completely coincided with those from PCR products. Nucleotide sequences of HV and LV and these amino acid sequences are shown below.

| Nucleotide sequence of cDNA of C40 heavy chain variable region (HV) | | |
|---|---|---|
| (from initiation codon (ATG) to the end of variable region)- | | |
| ATGAAGCACC TGTGGTTCTT CCTCCTGCTG GTGGCGGCTC CCAGATGGGT CCTGTCCCAG | 60 | (SEQ ID NO:9) |
| CTGCAGCTGC AGGAGTCGGG CCCAGGACTG GTGAAGCCTT CGGAGACCCT GTCCCTCACC | 120 | |
| TGCACTGTCT CTGGTGGTTC CATCAGCAGT AGTTTTTACT ACTGTGGCTG GATCCGCCAG | 180 | |
| CCCCCAGGGA AGGGGCTGGA GTGGATTGGG AGTATCTATT ATCGTGGGAG CACCTACTAC | 240 | |
| AACCCGTCCC TCAAGAGTCG AGTCACCATA TCCGTAGACA CGTCCAAGAA CCAGTTCTCC | 300 | |
| CTGAAGCTGA GCTCTGTGAC CGCCGCAGAC ACGGCTGTGT ATTACTGTGC GAGACGGGTT | 360 | |
| ACTATGGTTC GGGGAGTTAA GGGGGACTAC TTTGACTACT GGGGCCAGGG AACCCTGGTC | 420 | |
| ACCGTCTCCT CA 432 | | |
| Nucleotide sequence of cDNA of C40 light chain variable region (LV) | | |
| (from initiation codon (ATG) to the end of variable region)- | | |
| ATGAGGGTCC TCGCTCAGCT CCTGGGGCTC CTGCTGCTCT GTTTCCCAGG TGCCAGATGT | 60 | (SEQ ID NO:10) |
| GACATCCAGA TGACCCAGTC TCCATCCTCA CTGTCTGCAT CTGTAGGAGA CAGAGTCACC | 120 | |
| ATCACTTGTC GGGCGAGTCA GGGTATTAGC AGCTGGTTAG CCTGGTATCA GCAGAAACCA | 180 | |
| GAGAAAGTCC CTAAGTCCCT GATCTATGCT GCATCCAGTT TGCAAAGTGG GGTCCCATCA | 240 | |
| AGGTTCAGCG GCAGTGGATC TGGGACAGAT TTCACTCTCA CCATCAGCAG CCTGCAGCCT | 300 | |
| GAAGATTTTG CAACTTATTA CTGCCAACAG TATAATTATT ACCCGCTCAC TTTCGGCGGA | 360 | |
| GGGACCAAGG TGGAGATCAA ACGA 384 | | |
| Amino acid sequence of cDNA of C40 heavy chain variable region (HV) | | |
| (leader sequence (underlined) and variable region)- | | |
| MKHLWFFLLL VAAPRWVLSQ LQLQESGPGL VKPSETLSLT CTVSGGSISS SFYYCGWIRQ | 60 | (SEQ ID NO:11) |
| PPGKGLEWIG SIYYRGSTYY NPSLKSRVTI SVDTSKNQFS LKLSSVTAAD TAVYYCARRV | 120 | |
| TMVRGVKGDY FDYWGQGTLV TVSS 144 | | |
| Amino acid sequence of cDNA of C40 light chain variable region (LV) | | |
| (leader sequence (underlined) and variable region) | | |
| MRVLAQLLGL LLLCFPGARC DIQMTQSPSS LSASVGDRVT ITCRASQGIS SWLAWYQQKP | 60 | (SEQ ID NO:12) |
| EKVPKSLIYA ASSLQSGVPS RFSGSGSGTD FTLTISSLQP EDFATYYCQQ YNYYPLTFGG | 120 | |
| GTKVEIKR 128 | | |

Generation of Expression Vector of Isotype-Changed Human Anti-M2 Antibody (C40-IgG1 Type):

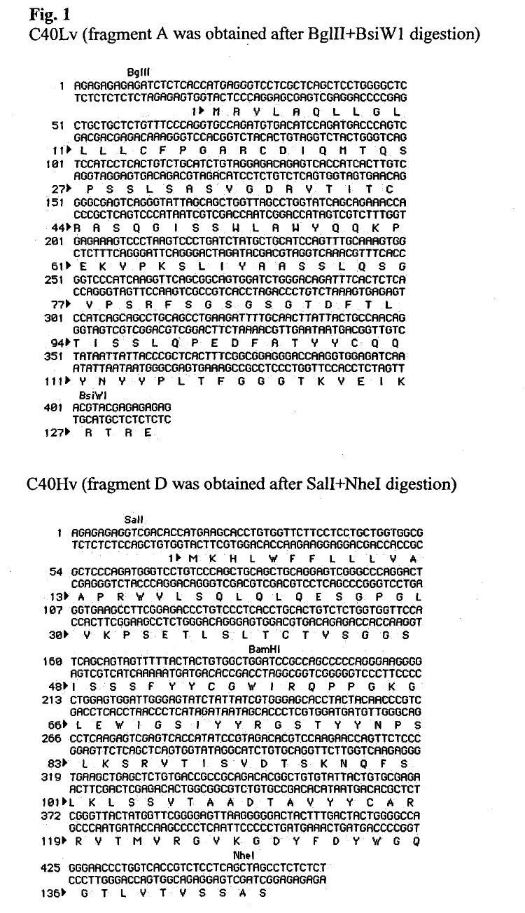

For making IgG1 type isotype-switched C40 antibody (the original isotype was IgG4), a new DNA vector was constructed. Briefly, the primer set for PCR of LV was designed to have sensitive region to restriction enzymes in the both sides of LV. The primer set used is M240L5BGL and M240L3BSI (Table 1), and construct plasmid of LV was used as a template. Purified PCR-amplified product of LV was subcloned into pGEM®-T Easy Vector System (Promega, Co., Ltd.). Nucleotide sequence of the insert was confirmed. The plasmid DNA was digested by two restriction enzymes, BglII and BsiWI, and 0.4 kilobases DNA insert (fragment A, see FIG. 1) was isolated and purified by the agarose gel electrophoresis.

Plasmid vector (IDEC Pharmaceuticals, CA, N5KG1-Val Lark (a modified vector of N5KG1 in U.S. Pat. No. 6,001,358)) was used as an expression vector for IgG1 production, which contains constant regions of both IgG1 light and heavy chains. The vector DNA was digested by the two enzymes, BglII and BsiWI, and subsequently treated with alkaline phosphatase (Takara Shuzo, Co., Ltd., Japan) for dephosphorylation of the end of the DNA. 8.9 kilobases DNA fragment (fragment B) was isolated by agarose gel electrophoresis and DNA purification kit.

Two DNA fragments, A and B were ligated with T4 DNA ligase (Takara Shuzo, Co., Ltd., Japan), and ligated construct (N5KG1_C40Lv) was electroporated into E.coli DH5α strain to generate transformants. Positive E.coli transformants were selected.

As the second step, HV was inserted into N5KG1_C40Lv DNA vector as follows: the DNA vector was digested by two DNA restriction enzymes, NheI and SalI, and subsequently dephosphorylated. 9.2 kilobases DNA fragment (fragment C) was isolated. Similarly to light chain construct, the primer set for PCR of HV was designed to have the sensitive region to restriction enzymes in the both sides of HV. The primer set used is M240H5SAL and M240H3NHE (Table 1), and construct plasmid of HV was used as a template. Purified PCR-amplified product of HV was subcloned into pGEM®-T Easy Vector System. Nucleotide sequence of the insert in the subcloned construct was confirmed. The plasmid DNA was digested by two restriction enzymes, NheI and SalI, and 0.44 kilobases DNA insert (fragment D, see FIG. 1) was isolated and purified after agarose gel electrophoresis.

Two DNA fragments, C and D were ligated with T4 DNA ligase, and ligated construct (N5KG1_M2C40) was electroporated into E.coli DH5a strain to generate transformant. Positive E.coli transformants were selected. This expression vector was purified, and nucleotide sequence of both LV and HV regions were confirmed. No mutations were introduced during the process.

TABLE 1

Synthesized DNA primers (SEQ ID NOS:13-30)

| | | | |
|---|---|---|---|
| No | Name | Sequence 5′ to 3′ | Length |

| | | | |
|---|---|---|---|
| 13 | IgG1 | TCTTGTCCACCTTGGTGTTGCTGGGCTTGTG | 31-mer |
| 14 | hk-2 | GTTGAAGCTCTTTGTGACGGGCGAGC | 26-mer |
| 15 | hh-4 | GGTGCCAGGGGGAAGACCGATGG | 23-mer |
| 16 | hk-5 | AGGCACACAACAGAGGCAGTTCCAGATTTC | 30-mer |
| 17 | hh-6 | GGTCCGGGAGATCATGAGGGTGTCCTT | 27-mer |
| 18 | SP6 | GATTTAGGTGACACTATAG | 19-mer |
| 19 | T7 | TAATACGACTCACTATAGGG | 20-mer |
| 20 | M240L-5BGL | AGAGAGAGAGATCTCTCACCATGAGGGTCCTCGCTCAGCTCCTG | 44-mer |
| 21 | M240L-3BSI | CTCTCTCTCGTACGTTTGATCTCCACCTTGGTCC | 34-mer |
| 22 | M240H5SAL | AGAGAGAGGTCGACACCATGAAGCACCTGTGGTTCTTCCT | 40-mer |
| 23 | M240H3NHE | CTCTCTCTGCTAGCTGAGGAGACGGTGACCAGG | 33-mer |
| 24 | SEQU1783 | GGTACGTGAACCGTGAGATCGCCTGGA | 27-mer |
| 25 | SEQU4618 | TCTATATAAGCAGAGCTGGGTACGTCC | 27-mer |
| 26 | hh-1 | CCAAGGGCCCATCGGTCTTCCCCCTGGCAC | 30-mer |
| 27 | CMVH903F | GACACCCTCATGATCTCCCGGACC | 24-mer |
| 28 | CMVHF1283 | CGACATCGCCGTGGAGTGGGAGAG | 24-mer |
| 29 | CMVHR1303 | TGTTCTCCGGCTGCCCATTGCTCT | 24-mer |
| 30 | hk-1 | TGGCTGCACCATCTGTCTTCATCTTC | 26-mer |

Generation of Expression Vector of Isotype-Changed Human Anti-M2 Antibody (IG4-Type C40):

For generation of DNA construct of IgG4 type C40, N5KG4PE DNA vector was used instead of N5KG1-Val Lark vector. This DNA vector contains constant regions of both light chain and heavy chains of IgG4. Procedure of generation of IgG4 vector of C40 was the same as that of IgG1-type C40.

Production of Recombinant Human Anti-M2 Antibody from CHO Cells:

For the production of recombinant antibody, generated DNA vector was transfected into host cells, and recombinant antibody was isolated from the supernatant of the transfected cells. Briefly, DNA vector was transfected into host cell dhfr-defective strain of Chinese Hamster Ovary cell (CHO cells, ATCC #CRL-9096) by electroporation. Twenty microgram of purified DNA expression vector, N5KG1_M2C40, was linearized by a DNA restriction enzyme, AscI, and the DNA was transfected into 4×10⁶ cells of CHO cells using Bio Rad electroporator (350V, 500 μF). The transfected cells were seeded in 96-well culture plate, and cells were cultured in the culture medium with Geneticin (Gibco-BRL) for selecting CHO cells containing the DNA vector. After the selection of several stable transfectant strains, high human IgG producers were screened by ELISA, and used for production of recombinant antibody.

Isolation and Purification of Recombinant Antibody Protein:

CHO cells expressing recombinant antibody were cultured with EX-CELL medium 325-PE (JRH Bioscience, Co., Ltd.). Ten liters of spent culture supernatant was used for purification of antibody protein as follows: The supernatant was applied to MabSelect Protein A columnn (Amersham Pharmacia Biotech, Co., Ltd.). For adsorption of antibody to protein A, phosphate-buffered saline (PBS) was used, and for elution 20 mM sodium citrate buffer and 50 mM sodium chloride (pH 2.7) was used. The pH of elution fraction was adjusted to 5.5 by addition of 50 mM sodium phosphate buffer (pH 7.0). Further purification of antibody was performed using SP Sepharose column (Amersham Pharmacia Biotech, Co., Ltd.), and PBS was used as an elution buffer.

Purified antibody was sterilized by filtering with Super Cup 100 Capsule membrane filter (0.22 μm diameter pore size). The concentration of the purified antibody was measured by spectrophotometry at 280 nm, in which 1 mg/ml of protein shows 1.4 OD at 280 nm. 17 mg of recombinant C40-IgG1 antibody was purified from 10 liters of CHO cell culture supernatant.

### Example 2

This example describes production and characterization of human and chimeric M2 monoclonal antibodies.

KM mice or HAC mice were immunized with synthetic M2 peptide based on the sequence derived from the M2 extracellular domain conjugated to KLH or BSA as a carrier. Most of the mice responded to M2 antigen with high titer as detected by ELISA with M2 peptide as coating antigen. Several anti-M2 human monoclonal antibodies were generated by fusion of splenocytes from 6 high responders with myeloma cells. Twelve monoclonal antibodies were obtained (denoted nos. 2074, C40, L17, L30, L40, L66, N547, S212, S80, S900, F1, and F2), that reacted to M2 peptide and/or M2-BSA conjugates, but did not respond to BSA, KLH (carriers for immunization), mGAD (a synthetic irrelevant peptide derived from mouse Glutamic Acid Decarboxylase (GAD), amino acids 246 to 266) as shown in Table 2. The coding sequences of variable regions of immunoglobulin heavy and light chains were cloned from the original C40 gene, and isotype-changed recombinant antibodies, C40G1 (IgG 1) and C40G4 (IgG4), were obtained using a CHO cell expression system (Example 1).

The human/mouse chimera monoclonal antibody no. 2074 and fully human antibodies C40G1, S212, S80, S900, N547, L66, F1, and F2 are IgG1 isotype. C40 is IgG4 isotype, L40 is IgG3 isotype and antibodies L17 and L30 are IgG2 isotypes (Table 2).

TABLE 2
The characters of anti-M2 human monoclonal antibodies derived from trans-
chromosome mouse.

| | | | | M2 on | | | | |
|---|---|---|---|---|---|---|---|---|
| | | Light | M2 | infected | | | | |
| mAbs | isotypes | chain | peptide* | cells** | BAS | OVA | KLH | mGAD*** |
| C40 | IgG4 | Kappa | +¹ | + | −² | − | − | − |
| C40G1 | IgG1 | Kappa | + | + | − | − | − | − |
| L17 | IgG2 | Lambda | + | + | − | − | − | − |
| L30 | IgG2 | Lambda | + | + | − | − | − | − |
| L40 | IgG3 | Lambda | + | + | − | − | − | − |
| L66 | IgG1 | Lambda | + | + | − | − | − | − |
| N547 | IgG1 | Lambda | + | + | − | − | − | − |
| S212 | IgG1 | Lambda | + | + | − | − | − | − |
| S80 | IgG1 | Lambda | + | + | − | − | − | − |
| S900 | IgG1 | Lambda | + | + | − | − | − | − |
| F1 | IgG1 | Kappa | + | + | − | − | − | − |
| F2 | IgG1 | Kappa | + | + | − | − | − | − |

*Most common extracellular portion of M2 protein; the sequence is: SLLTEVETPIRNEWGCRCNDSSD (SEQ ID NO: 1)
**Binding to M2 expressed on A/PR/8/34 and A/HK/8/68 infected MDCK cells at 10 μg/ml
***A synthetic peptide derived from mouse Glutamic Acid Decarboxylase (mGAD), at position from 246 to 266
¹positive was defined as 2-fold higher than negative control at OD₄₅₀ nm
²negative was below 0.1 at OD₄₅₀ nm

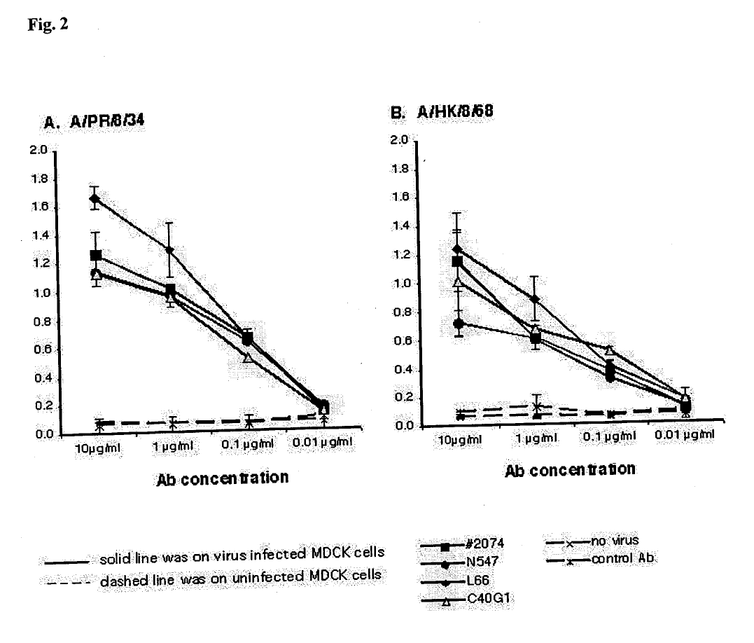

All antibodies recognized M2 expressed on MDCK cells infected with either influenza A/PR/8/34 or A/HK/8/68 strains indicating that the antibodies recognize the native form of M2 expressed by the two different strains even though the sequences of the extracellular domains are slightly different. (FIG. 2). Moreover, antibodies binding to the infected cells were specifically inhibited when M2 peptide was presented (representative data shown in Table 3).

The extracellular portion of the M2 sequence between these two virus strains differs by a single amino acid: a substitution of an aspartic acid to glycine at position 20 in the extracellular portion of M2 in the A/PR/8/34 strain. The sequence derived from A/HK/8/68, so-called universal M2 extracellular portion, is shared among most influenza strains (Neirynck et al., Nature Med. 5:1157 (1999)). However, this one mutation abolished binding by a different mouse anti-M2 monoclonal antibody, 14C2 (Gerhard et. al. Immunological Rev. 159:95 (1997)).

The reactivity of antibody nos. 2074, N547, L66, L17, C40G1, was comparable and approximately 3 to 5-fold greater than those of antibodies C40G4, S212, and S80, and more than 100-fold greater than F1 and F2 towards A/PR/8/34 virus strain (FIG. 2 and Table 4).

Regarding response to M2 on A/HK/8/68 infected cells, S212, S80, S900, F1 and F2 was approximately 100-fold less than the other antibodies (Table 4). As expected, isotype matched irrelevant human anti-HSA antibody (anti-human serum albumin) did not show any reactivity.

TABLE 3
Specific inhibition of mAbs binding
on M2 on viral infected MDCK cells in the
presence of 20 μg/ml M2 peptide.

| MAbs* | A/PR/8/34 | M2 | OD₄₅₀ |
|---|---|---|---|
| 2074 | − | − | 0.051 |
| | + | − | 0.904 |
| | + | + | 0.142 |
| N547 | − | − | 0.065 |
| | + | − | 0.504 |
| | + | + | 0.062 |
| L66 | − | − | 0.051 |
| | + | − | 0.931 |
| | + | + | 0.113 |
| C40G1 | − | − | 0.051 |
| | + | − | 0.799 |
| | + | + | 0.195 |

*All antibodies are used at 1 μg/ml concentration.

TABLE 4
Binding capability of anti-M2 antibody to
native M2 on MDCK cells infected with two
influenza A virus strains.

| | EC₅₀ (μg/ml) of Abs* | |
|---|---|---|
| | to M2 on MDCK cells infected by | |
| mAbs | A/PR/8/34 | A/HK/8/68 |
| 2074 | 0.0891 | 0.1873 |
| C40G1 | 0.1826 | 0.0971 |
| C40G4 | 0.3007 | 0.8414 |
| S212 | 0.5001 | >10** |
| S80 | 0.2176 | >10** |
| S900 | 0.2063 | >10** |
| N547 | 0.1042 | 0.4661 |
| L17 | 0.1511 | 0.5968 |
| L30 | 0.1747 | 3.4914 |
| L66 | 0.1169 | 0.2289 |
| F1 | >10** | >10** |
| F2 | >10** | >10** |

*OD₄₅₀ of no. 2074 at 10 μg/ml dose was set as 100% for EC₅₀ calculation. The background is below 0.1.
**These Abs are very weak binder, and the OD₄₅₀ at 10 μg/ml is even less than half of the OD₄₅₀ of no. 2074 antibody at the same concentration.

Binding activity of anti-M2 antibodies to mutant M2 peptides was analyzed in an ELISA assay using eight different M2 peptides (SEQ ID NO: 1-8, Table 5) that have been reported in influenza A virus. Anti-M2 antibody nos. 2074, C40, C40G1, L66 and N547 exhibited binding activity to the M2 peptides as well as the original M2 peptide (Table 6). Especially, C40G1 and N547 bound to all eight M2 peptides used in the study.

A/HK/8/68 and A/PR/8/34 virus strains have peptide sequences listed as SEQ ID NO: 1 and 9, respectively, in M2 protein. Since the above mentioned anti-M2 antibodies bind to cell surface M2 protein in MDCK cells infected by either of these two virus strains, these antibodies can also bind to the M2 peptide set forth as SEQ ID NO: 9 (i.e. M2G, Table 5).

These results indicate that invention anti-M2 antibodies have broad specificity to bind various M2 mutant peptides, which are observed in mutant influenza A virus strains.

TABLE 5

Sequences of M2 analogs

| | | |
|---|---|---|
| M2 analogs | Sequence | SEQ ID NO |

| | | |
|---|---|---|
| M2 | S L L T E V E T P I R N E W G C R C N D S S D | 1 |
| M2K | S L L T E V E T P I R N E W G C KC N D S S D | 2 |
| M2P | S L PT E V E T P I R N E W G C R C N D S S D | 3 |
| M2SG | S L L T E V E T P I R SE W G C R C N D S GD | 4 |
| M2FG | S FL T E V E T P I R N E W G C R C N GS S D | 5 |
| M2EG | S L L T E V E T P I R N E W EC R C N GS S D | 6 |
| M2TGS | S L L T E V E T P TR N GW G C R C SD S S D | 7 |
| M2TGE | S L L T E V E TP T R N GW EC R C N D S S D | 8 |
| M2G | S L L T E V E T P I R N E W G C R C N GS S D | 9 |

Underlined bold characters are the regions of mutation compared to the original M2 sequence (SEQ ID NO: 1).

TABLE 6
Broad binding activity of anti-M2 antibodies to M2 analogs

| mAbs | M2* | M2TGE | M2EG | M2TSG | M2K | M2SG | M2P | M2FG |
|---|---|---|---|---|---|---|---|---|
| 2074 | +¹ | + | + | + | + | + | −² | + |
| C40 | + | + | + | + | + | + | + | + |
| C40G1 | + | + | + | + | + | + | + | + |
| L66 | + | + | + | + | + | + | − | − |
| N547 | + | + | + | + | + | + | + | + |

*Most common extracellular portion of M2 protein is: SLLTEVETPIRNEWGCRCNDSSD (SEQ ID NO: 1)
¹positive was defined as 2-fold higher than negative control at OD₄₅₀ nm
²negative was below 0.1 at OD₄₅₀ nm

### Example 3

This example describes animal model studies indicating that administering an M2 monoclonal antibody of the invention before and after the animal is infected with influenza virus protects against a lethal challenge of virus.

In vivo Efficacy of Anti-M2 mAb for Prophylaxis Treatment (Prior to Virus Infection) in a Mouse Influenza A Virus Model:

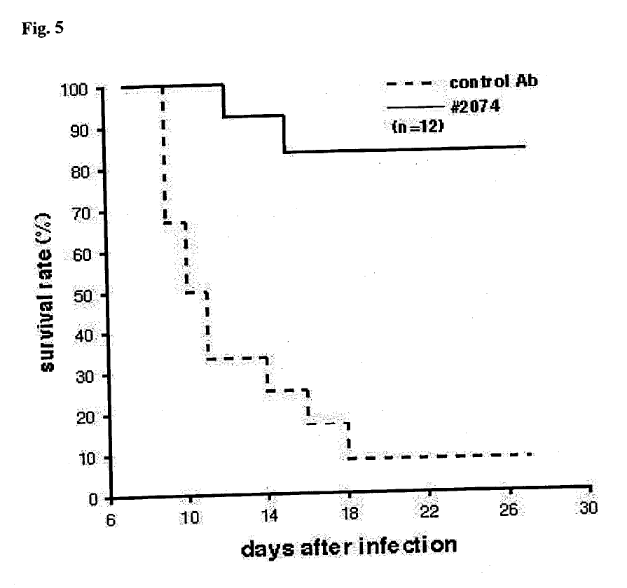

To evaluate the efficacy of anti-M2 human/mouse chimera monoclonal antibody in an animal model, antibody no. 2074 was administered at a dose of 200 μg/mouse intraperitoneally to female C57BL/6J mice (8˜10 weeks old). One day after initiation of treatment, anesthetized mice (15 μl/g of Avertin (1:1 w/v of 2,2,2 tribromoethanol:tert-amyl-OH, Sigma, St. Louis, Mo.)) were infected with 30 μl (300 pfU /30 μl) of a lethal dose of influenza A/PR/8/34 (ATCC) intranasally. Two days after infection, the mice received another dose of no. 2074 antibody (200 μg/mouse) intraperitoneally. Mice were observed daily for 27 days for survival analysis. The surviving mice were sacrificed after that time and the lungs were removed for detection of virus and histological analysis. The survival analysis is shown in FIG. 5. As a control, an isotype matched human monoclonal anti-HSA IgG1 antibody generated from a KM mouse was used (Kirin, Japan). The results are illustrated in Table 5.

In the control group, 11 of 12 mice died within 18 days post infection. In contrast, anti-M2 antibody no. 2074 treated mice were significantly protected. Ten of 12 mice were still alive over the 27-day period of observation. The surviving mice (10 from the anti-M2 treated group and 1 from the control group) were sacrificed at day 27 after infection and the lungs were removed for viral titer and tissue analysis. No detectable virus from the lungs of the mice from either group was found by a viral plaque assay, while for the positive control, the titer of A/HK/8/68 virus was 5.95×10³ pfU/ml (Table 5). This data indicates that administration of anti-M2 antibody can prevent and increase in viral titer in the lung in mice, and eventually facilitate viral clearance in the mouse body.

TABLE 5
Viral titer of the lungs from mice at day 27 after A/PR/8/34 infection.

| Samples | Dilution | No of plaques | pfu / ml |
|---|---|---|---|
| 1-L1* | 10⁻¹ | 0 | <50** |
| 1-l2 | 10⁻¹ | 0 | <50 |
| 1-L3 | 10⁻¹ | 0 | <50 |
| 1-L4 | 10⁻¹ | 0 | <50 |
| 1-L5 | 10⁻¹ | 0 | <50 |
| 1-L11 | 10⁻¹ | 0 | <50 |
| A/HK/8/68*** | 10⁻³ | 59.5 | 5.95 × 10³ |

*Lung homogenates from A/PR/8/68 infected mice.
L1 to L5: samples from anti-M2 antibody treated group.
L11: sample from isotype matched antibody treated group. (control)
**Threshold of virus detection is 50 pfu/ml.
***Virus used as positive control for the assay.

In Vivo Efficacy of Anti-M2 mAb for Therapeutic Treatment (After Virus Infection) in a Mouse Influenza A Virus Model.

Anesthetized female C57BL/6J mice (8˜10 weeks old) were infected with 30 μl of a lethal dose of influenza A/PR/8/34 (ATCC) intranasally. Anesthetization was performed using Avertin as described above. Mice were observed daily for 24 days for survival analysis.

To evaluate the efficacy of the anti-M2 monoclonal antibodies for therapeutic treatment of influenza virus, the antibody was administered after virus infection. Two and four days after a lethal dose virus challenge of influenza A/PR/8/34 was given to C57BL/6J mice, anti-M2 antibody no. 2074 was administered at 200 μg/mouse in each time by intraperitoneal injection (12 mice in total). The control group (total 12 mice) received isotype matched irrelevant human monoclonal antibody (anti-HSA (IgG1) from Kirin Brewery Co., Ltd., Japan)).

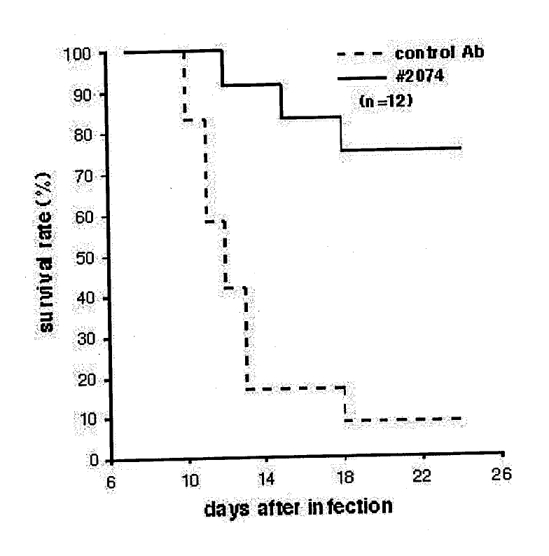

In the control group, 11 of 12 mice died within 18 days post infection (FIG. 6). In the antibody no. 2074 group, nine of 12 mice survived virus challenge at day 24. Thus, anti-M2 human/mouse chimera monoclonal antibody no. 2074 significantly increased survival of mice infected with A/PR/8/34 virus.

The data indicates that anti-M2 antibody is effective by administration even after virus infection. This implies the antibody can be used not only for prophylaxis but therapeutic use.

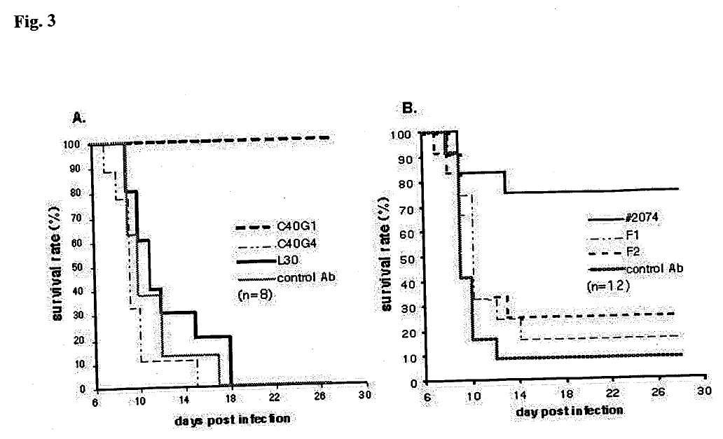

Another two sets of evaluation study were performed. A lethal dose virus challenge of influenza A/PR/8/34 was given to C57BL/6J mice, and one, two and three days later, anti-M2 antibodies C40G1, C40G4, L30, F1, F2 and no. 2074 (as a positive control) were administered at 200 μg/mouse in each time by intraperitoneal injection (n=8 or 12 mice in each group). The control group (total of 8 or 12 mice) received anti-HSA specific human IgG1 antibody injection. L30, C40G4, F1 and F2 antibodies did not prolong survival of virus infected mice compared with control group (FIGS. 3A, B). In contrast, C40G1 antibody showed clear protection from the viral challenge, and all mice in this group were still alive even after 30 days post infection (FIG. 3A).

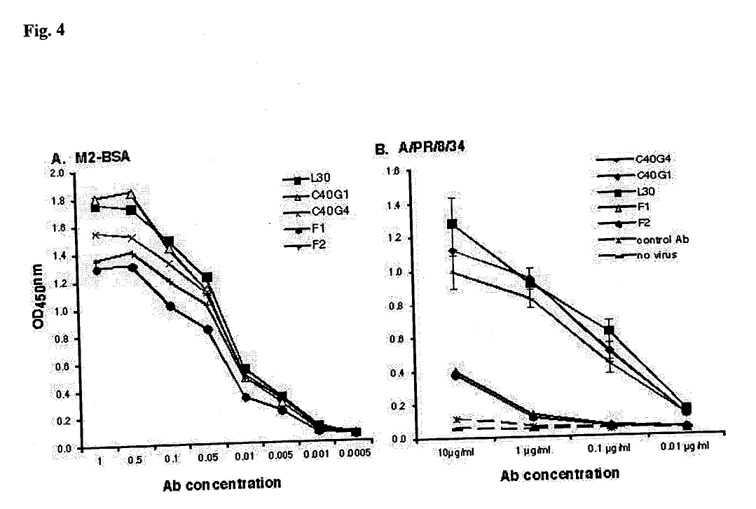

Binding affinity of C40G1, L30 and C40G4 antibodies to either M2 expressed on A/PR/8/34 infected cells (FIG. 4B, Table 4) or M2-BSA conjugate (FIG. 4A) were not significantly different among each other. C40G1 and C40G4 have the same antigen binding site, since both of them came from C40 antibody. Since L30 (IgG2) and C40G4 (IgG4) did not show protection from virus challenge whereas C40G1 did significantly protect, IgG1 type antibody is potentially a better candidate for in vivo use. In contrast, F1 and F2 antibodies bound poorly to M2 on viral infected cells although these antibodies bind to M2-BSA conjugate well (FIGS. 4A, B, Table 4). The poor binding of F1 and F2 antibodies to M2 on viral infected cells may account for the lack of protective effect in vivo.

What is claimed:

1. An antibody that specifically binds to influenza protein M2, wherein the antibody comprises a human, humanized or chimeric monoclonal antibody.

2. The antibody of claim 1, wherein the antibody binds to at least a part of the M2 extracellular domain or a subsequence of the M2 extracellular domain.

3. The antibody of claim 2, wherein the extracellular domain comprises the amino acid sequence SLLTEVETPIRNEWGCRCNDSSD (SEQ ID NO: 1).

4. The antibody of claim 3, wherein the subsequence comprises four or more contiguous amino acids in SLLTEVETPIRNEWGCRCNDSSD (SEQ ID NO: 1).

5. The antibody of claim 2, wherein the extracellular domain comprises an amino acid substitution, insertion, deletion or addition of the amino acid sequence SLLTEVETPIRNEWGCRCNDSSD (SEQ ID NO: 1).

6. The antibody of claim 5, wherein the extracellular domain comprises a sequence having an amino acid substitution selected from: SLLTEVETPIRNEWGCKCNDSSD, SLPTEVETPIRNEWGCRCNDSSD, SLLTEVETPIRSEWGCRCNDSGD, SFLTEVETPIRNEWGCRCNGSSD, SLLTEVETPIRNEWECRCNGSSD, SLLTEVETPTRNGWGCRCSDSSD, or SLLTEVETPIRNGWECRCNDSSD (SEQ ID NOS: 2-8, respectively.

7. The antibody of claim 5, wherein the subsequence comprises four or more contiguous amino acids in any of SLLTEVETPIRNEWGCKCNDSSD, SLPTEVETPIRNEWGCRCNDSSD, SLLTEVETPIRSEWGCRCNDSGD, SFLTEVETPIRNEWGCRCNGSSD, SLLTEVETPIRNEWECRCNGSSD, SLLTEVETPTRNGWGCRCSDSSD, or SLLTEVETPIRNGWECRCNDSSD (SEQ ID NOS: 2-8, respectively).

8. The antibody of claim 1, wherein the antibody is selected from IgG, IgA, IgM IgE, and IgD isotypes

9. The antibody of claim 8, wherein the isotype is selected from IgG₁, IgG₂, IgG₃ and IgG₄.

10. The antibody of claim 1, wherein the antibody is produced by a hybridoma or a CHO cell line denoted as no. 2074 (ATCC Deposit No. PTA-4025), 161(ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

11. The antibody of claim 1, wherein the antibody has the binding specificity of an antibody produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

12. The antibody of claim 1, wherein the antibody has the same or substantially the same binding affinity as an antibody produced by a hybridoma or a CHO cell line denoted as no. 2074 (ATCC Deposit No. PTA-4025), 161(ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

13. The antibody of claim 12, wherein the affinity is within about 5 to 100 fold of the reference antibody, or within about 5 to 5000 fold of the reference antibody.

14. The antibody of claim 1, wherein the antibody inhibits virus infection of a cell, virus proliferation or virus replication in vitro or in vivo.

15. The antibody of claim 1, wherein the antibody inhibits influenza binding of a cell in vitro or in vivo.

16. The antibody of claim 1, wherein the antibody inhibits increases in virus titer, decreases virus titre, decreases virus replication or proliferation, or decreases one or more symptoms or complications associated with virus infection in a subject.

17. The antibody of claim 1, wherein the antibody inhibits increases in virus titer, decreases virus titre, decreases virus replication or proliferation, or decreases one or more symptoms or complications associated with virus infection in a subject after the subject has been exposed to or infected with the virus.

18. The antibody of claims 16 or 17, wherein the symptoms or complications are selected from chills, fever, cough, sore throat, nasal congestion, sinus congestion, nasal infection, sinus infection, body ache, head ache, fatigue, pneumonia, bronchitis, ear infection, ear ache and death.

19. The antibody of claims 16 or 17, wherein the antibody has the binding specificity or the binding affinity of an antibody produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

20. The antibody of claim 1, wherein the antibody inhibits virus infection of a subject, and the antibody has the same or substantially the same binding specificity or the binding affinity of an antibody produced by a hybridoma or a CHO cell line denoted as no. 2074 (ATCC Deposit No. PTA-4025), 161(ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

21. The antibody of claim 1, wherein the antibody decreases susceptibility of a subject to virus infection.

22. The antibody of claim 21, wherein the antibody has the binding specificity or the same or substantially the same binding affinity of an antibody produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

23. The antibody of claim 1, wherein the influenza virus comprises influenza A virus.

24. The antibody of claim 23, wherein the influenza virus comprises A/PR/34, A/HK8/68, H1N1, H2N2, H3N2, H5N1, H9N2, H2N1, H4N6, H6N2, H7N2, H7N3, H4N8, H5N2, H2N3, H11N9, H3N8, H1N2, H11N2, H11N9, H7N7, H2N3, H6N1, H13N6, H7N1, H11N1, H7N2 and H5N3.

25. The antibody of claim 1, wherein the antibody has an EC₅₀ less than 3.0 μg/ml for inhibiting influenza virus infection of MDCK cells, as determined by a cell based-ELISA assay.

26. The antibody of claim 25, wherein the influenza virus comprises A/PR/8/34, A/HK8/68, H1N1, H2N2, H3N2, H5N1, H9N2, H2N1, H4N6, H6N2, H7N2, H7N3, H4N8, H5N2, H2N3, H11N9, H3N8, H1N2, H11N2, H11N9, H7N7, H2N3, H6N1, H13N6, H7N1, H11N1, H7N2 and H5N3.

27. The antibody of claim 25, wherein the antibody has the binding specificity or the same or substantially the same binding affinity of an antibody produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

28. The antibody of claim 1, wherein the antibody has an EC₅₀ less than 3.0 μg/ml for inhibiting M2 binding to MDCK cells, as determined by a cell based-ELISA assay.

29. The antibody of claim 28, wherein the antibody has the binding specificity or the same or substantially the same binding affinity of an antibody produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

30. The antibody of claim 1, wherein the antibody specifically binds to two or more influenza virus strains or isolates.

31. The antibody of claim 1, wherein the antibody specifically binds to two or more M2 proteins having a different sequence.

32. The antibody of claim 31, wherein the M2 protein comprises the extracellular domain.

33. The antibody of claim 32, wherein the M2 protein extracellular domain is selected from SLLTEVETPIRNEWGCRCNDSSD, SLLTEVETPIRNEWGCKCNDSSD, SLPTEVETPIRNEWGCRCNDSSD, SLLTEVETPIRSEWGCRCNDSGD, SFLTEVETPIRNEWGCRCNGSSD, SLLTEVETPIRNEWECRCNGSSD, SLLTEVETPTRNGWGCRCSDSSD, or SLLTEVETPIRNGWECRCNDSSD (SEQ ID NOS: 1-8, respectively).

34. An amino acid subsequence of the antibody of claim 1.

35. The antibody of claim 34, wherein the subsequence has the binding specificity or binding affinity of the antibody of claim 1.

36. The antibody of claim 34, wherein the subsequence is selected from heavy and light chain variable regions (V_{H} and V_{L}), Fab, Fab′, (Fab′)₂, Fv, Fd, scFv, and sdFv.

37. The antibody of claim 1, wherein the antibody comprises an antibody multimer.

38. The antibody of claim 1 or a subsequence thereof, further comprising one or more heterologous domains.

39. The antibody of claim 38, wherein the heterologous domain comprises an amino acid sequence.

40. The antibody of claim 38, wherein the heterologous domain comprises a binding protein, an enzyme activity, a drug, an antiviral, a toxin, an immune-modulator, a detectable moiety or a tag

41. A bispecific or bifunctional antibody of claim 1.

42. A host cell that expresses an antibody of claim 1.

43. The cell of claim 42, wherein the antibody has the binding specificity or the same or substantially the same binding affinity of an antibody produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

44. The cell of claim 42, wherein the antibody is produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

45. The cell of claim 42, wherein the cell is bacteria, yeast, plant or animal.

46. A non-human transgenic animal or plant that expresses an antibody of claim 1.

47. A nucleic acid encoding an antibody produced by a hybridoma or a CHO cell line denoted as no. 2074 (ATCC Deposit No. PTA-4025), 161(ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

48. The nucleic acid of claim 47, further comprising a vector.

49. The antibody of claim 1, further comprising an antiviral agent.

50. The antibody of claim 1, further comprising an agent that inhibits one or more symptoms or complications associated with influenza virus infection.

51. The antibody of claim 50, wherein the symptoms or complications are selected from chills, fever, cough, sore throat, nasal congestion, sinus congestion, nasal infection, sinus infection, body ache, head ache, fatigue, pneumonia, bronchitis, ear infection, ear ache and death.

52. A pharmaceutical composition comprising the antibody of claim 1, and a pharmaceutically acceptable carrier or excipient.

53. A kit comprising the antibody of claim 1, and instructions for treating, inhibiting, preventing or decreasing susceptibility of infection of a subject by one or more influenza virus strains or isolates.

54. The kit of claim 53, further comprising an article of manufacture for delivery of the antibody into a mucosal tissue.

55. The kit of claim 53, wherein the article of manufacture comprises an inhaler, aerosol, spray or squeeze bottle suitable for inhalation or nasal administration to a subject.

56. The kit of claim 53, wherein the mucosal tissue comprises nasal passages, sinuses, mouth, throat, larynx or lungs.

57. The kit of claim 53, further comprising an antiviral agent.

58. The kit of claim 53, further comprising an agent that inhibits one or more symptoms or complications associated with influenza virus infection.

59. A method for treating influenza virus infection of a subject, comprising administering to the subject an amount of a human, humanized or chimeric monoclonal antibody that specifically binds influenza M2 effective to treat influenza virus infection of the subject.

60. The method of claim 59, wherein the antibody is administered prior to, substantially contemporaneously with or following infection of the subject.

61. The method of claim 59, wherein the antibody is administered substantially contemporaneously with or following infection of the subject.

62. The method of claim 59, wherein the administration provides a therapeutic benefit.

63. The method of claim 59, wherein the therapeutic benefit comprises inhibiting increases in virus titer, decreasing virus titer, inhibiting increases in virus replication, decreasing virus replication, inhibiting increases in virus proliferation or decreasing virus proliferation, or decreasing one or more symptoms or complications associated with virus infection in a subject.

64. The method of claim 63, wherein the symptoms or complications are selected from chills, fever, cough, sore throat, nasal congestion, sinus congestion, nasal infection, sinus infection, body ache, head ache, fatigue, pneumonia, bronchitis, ear infection, ear ache and death.

65. The method of claim 59, wherein the therapeutic benefit comprises hastening a subject's recovery from influenza virus infection.

66. The method of claim 59, wherein the antibody has the binding specificity or the same or substantially the same binding affinity of an antibody produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

67. The method of claim 59, wherein the antibody is produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

68. The method of claim 59, wherein the antibody has an EC₅₀ less than 3.0 μg/ml for inhibiting influenza virus infection of MDCK cells, as determined by a cell based-ELISA assay.

69. The method of claim 59, wherein the influenza strain comprises A/PR/8/34, A/HK/8/68, H1N1, H2N2, H3N2, H5N1, H9N2, H2N1, H4N6, H6N2, H7N2, H7N3, H4N8, H5N2, H2N3, H11N9, H3N8, H1N2, H11N2, H11N9, H7N7, H2N3, H6N1, H13N6, H7N1, H11N1, H7N2 and H5N3.

70. A method for inhibiting infection of a subject by one or more influenza virus strains or isolates comprising administering to the subject an amount of a human, humanized or chimeric antibody that specifically binds influenza M2 effective to inhibit infection of the subject by one or more influenza virus strains or isolates.

71. The method of claim 70, wherein the antibody is administered prior to, substantially contemporaneously with or following virus infection of the subject.

72. The method of claim 70, wherein the antibody is administered substantially contemporaneously with or following virus infection of the subject.

73. The method of claim 70, wherein the administration provides a therapeutic benefit.

74. The method of claim 70, wherein the therapeutic benefit comprises protecting the subject from virus infection or decreasing susceptibility of the subject from virus infection.

75. The method of claim 70, wherein the antibody has the binding specificity or the same or substantially the same binding affinity of an antibody produced by a hybridoma or a CHO cell line denoted as no. 2074 (ATCC Deposit No. PTA-4025), 161 (ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

76. The method of claim 70, wherein the antibody is produced by a hybridoma or a CHO cell line denoted as no. 2074 (ATCC Deposit No. PTA-4025), 161(ATCC Deposit No. PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

77. The method of claim 70, wherein the antibody has an EC₅₀ less than 3.0 μg/ml for inhibiting influenza virus infection of MDCK cells, as determined by a cell based-ELISA assay.

78. The method of claim 70, wherein the influenza strain comprises AIPR/8/34, A/HK/8/68, H1N1, H2N2, H3N2, H5N1, H9N2, H2N1, H4N6, H6N2, H7N2, H7N3, H4N8, H5N2, H2N3, H11N9, H3N8, H1N2, H11N2, H11N9, H7N7, H2N3, H6N1, H13N6, H7N1, H11N1, H7N2 and H5N3.

79. The antibody of claim 1, wherein the antibody comprises heavy-chain variable sequence and light-chain variable sequence of the antibody produced by a hybridoma or a CHO cell line denoted as no. 2074 (ATCC PTA-4025), 161 (ATCC PTA-4026), N547 (ATCC Deposit No.; American Type Culture Collection, Manassas, Va., USA, received by ATCC on Mar. 11, 2003), L66 (ATCC Deposit No.; American Type Culture Collection, Manassas, Va., USA, received by ATCC on Mar. 11, 2003), C40G1 (ATCC Deposit No.; American Type Culture Collection, Manassas, Va., USA, received by ATCC on Mar. 11, 2003), and L17 (ATCC Deposit No.; American Type Culture Collection, Manassas, Va., USA).

80. The antibody of claim 1, wherein the antibody comprises heavy-chain variable sequence and light-chain variable sequence encoded by the nucleic acid sequences set forth as SEQ ID NO: 9 and SEQ ID NO: 10, or a nucleic acid sequence degenerate with respect to SEQ ID NO: 9 and SEQ ID NO: 10.

81. The antibody of claim 1, wherein the antibody comprises heavy-chain variable sequence and light-chain variable sequence as set forth in SEQ ID NO: 11 and SEQ ID NO: 12.

82. The antibody of any of claims 79 to 81, wherein the antibody comprises a human IgG1 subtype.
